(19) United States
(12) Patent Application Publication
(10) Pub. No.: US 2005/0075664 A1
(43) Pub. Date: Apr. 7, 2005

(54) Surgical instrument

(75) Inventors: Toru Nagase, Tokyo (JP); Kazunori Taniguchi, Tokyo (JP)

Correspondence Address: SCULLY SCOTT MURPHY & PRESSER, PC, 400 GARDEN CITY PLAZA, GARDEN CITY, NY, 11530, US

(73) Assignee: OLYMPUS CORPORATION, TOKYO (JP)

(21) Appl. No.: 10/678,503

(22) Filed: Oct. 3, 2003

(51) Int. Cl. A61B 17/28

(52) U.S. Cl. 606/205

(57) ABSTRACT

The surgical instrument comprises: an insertion portion; a treatment unit which is provided on the tip portion of the insertion portion and which is capable of turning as to the shaft of the insertion portion and capable of opening and closing; an operating unit provided at the base portion of the insertion portion and which is capable of turning as to the shaft of the insertion portion and capable of opening and closing; a supporting member linking the treatment unit and the operating unit; and a plurality of driving rods arrayed in parallel or generally in parallel with the supporting member, so as to be capable of advancing and retreating; wherein the opening/closing operating mechanism of the treatment unit and the opening/closing operating mechanism of the operating unit are in a parallel mapping relation following the insertion portion.

### BACKGROUND OF THE INVENTION

1. Field of the Invention

The present invention relates to a surgical instrument, more specifically an instrument used in surgical operations and in particular in surgical operations using an endoscope, which is a surgical instrument such as a multiple-degree-of-freedom forceps device or the like configured so as to turn and open and close a treatment unit provided on the tip portion of an insertion portion, by a surgeon holding and operating an operating unit provided on the base portion of the insertion portion.

2. Description of the Related Art

Conventionally, various proposals have been made regarding surgical instruments used for surgical operations, such as U.S. Pat. No. 5,275,608, U.S. Pat. No. 5,702,408, U.S. Pat. No. 5,383,888, and U.S. Pat. No. 4,763,669, for example.

The means according to the above U.S. Pat. No. 5,275,608 are configured of a shaft, a treatment unit capable of opening and closing, which is provided on the tip portion of this shaft, and a handle provided on the base portion of the shaft, configured such that the treatment unit can be not only opened and closed but also a pair of treatment members can be turned within the same plane by operating the handle.

Also, the means according to the above U.S. Pat. No. 5,702,408 comprises a first link having a lever-like form, second and third links connected to the first link, with the second and third links being mutually movable inwards, and a fourth link connected to both link ends. The treatment unit and operating unit are configured so as to assume the same angle due to a parallel link mechanism wherein turning the first link in a first direction causes the fourth link to also turn in the first direction.

Also, the means according to the above U.S. Pat. No. 5,383,888 are configured of a shaft, a treatment unit capable of opening and closing and also turning, which is provided on the tip portion of this shaft, and a handle provided on the base portion of the shaft. This is configured such that the treatment unit can be opened and closed with the handle, and also the treatment unit can be turned by advancing or retracting operating rod with a lever provided to the handle side.

Also, the means according to the above U.S. Pat. No. 4,763,669 are configured of a shaft, a treatment unit capable of opening and closing and also turning, which is provided on the tip portion of this shaft, and a handle provided on the base portion of the shaft. This is configured such that the treatment unit can be opened and closed with the handle, and also the treatment unit can be turned by advancing or retracting push rods with a lever provided to the handle side.

### SUMMARY OF THE INVENTION

In brief, the surgical instrument according to the present invention comprises: an insertion portion; a treatment unit which is provided on the tip portion of the insertion portion and which is capable of turning as to the shaft of the insertion portion and capable of opening and closing; an operating unit provided at the base portion of the insertion portion and which is capable of turning as to the shaft of the insertion portion and capable of opening and closing; a supporting member linking the treatment unit and the operating unit; and multiple driving rods arrayed in parallel or generally in parallel with the supporting member, so as to be capable of advancing and retreating; wherein the opening/closing operating means of the treatment unit and the opening/closing operating means of the operating unit are in a parallel mapping relation following the insertion portion.

Advantages of the present invention will become clearer from the following description.

### BRIEF DESCRIPTION OF THE DRAWINGS

FIG. 1 is a perspective view illustrating the general overall configuration of a surgical instrument according to a first embodiment of the present invention;

FIG. 2 is an enlarged perspective view of principal members illustrating around the treatment unit of the surgical instrument shown in FIG. 1, primarily showing the upper side of the treatment unit;

FIG. 3 is an enlarged perspective view of principal members illustrating around the treatment unit of the surgical instrument shown in FIG. 1, primarily showing the lower side of the treatment unit;

FIG. 4 is an enlarged perspective view of principal members illustrating around the treatment unit of the surgical instrument shown in FIG. 1, primarily showing the upper side with the treatment unit disposed in an opened state;

FIG. 5 is an enlarged perspective view of principal members illustrating around the operating unit of the surgical instrument shown in FIG. 1, primarily showing the upper side of the operating unit;

FIG. 6 is an enlarged perspective view of principal members illustrating around the operating unit of the surgical instrument shown in FIG. 1, primarily showing the lower side of the operating unit;

FIG. 7 is an enlarged perspective view of principal members illustrating a state wherein a part of the members (the handle and a part of the guard) have been removed from the operating unit shown in FIG. 5;

FIG. 8 is an enlarged perspective view of principal members illustrating primarily the upper side in a state wherein a part of the members (the handle and the guard) have been removed from the operating unit shown in FIG. 5;

FIG. 9 is an enlarged perspective view of principal members illustrating primarily the lower side in a state wherein a part of the members (the handle and the guard) have been removed from the operating unit shown in FIG. 5;

FIG. 10 is an enlarged perspective view of principal members illustrating primarily the upper side in a state wherein further components have been removed from the operating unit from the state shown in FIG. 8;

FIG. 11 is an enlarged perspective view of principal members illustrating primarily the lower side in a state wherein further components have been removed from the operating unit from the state shown in FIG. 8;

FIG. 12 illustrates an operating form of the surgical instrument shown in FIG. 1, and is a side view with the treatment unit of the surgical instrument in a horizontal state;

FIG. 13 illustrates an operating form of the surgical instrument shown in FIG. 1, and is a plan view of the upper side with the treatment unit of the surgical instrument in a horizontal state;

FIG. 14 illustrates an operating form of the surgical instrument shown in FIG. 1, and is a plan view of the lower side with the treatment unit of the surgical instrument in a horizontal state;

FIG. 15 is a side view illustrating a state wherein the treatment unit in the state shown in FIG. 12 (a horizontal state) is turned upwards (arrow Y1);

FIG. 16 is a plan view of the upper side illustrating a state wherein the treatment unit in the state shown in FIG. 13 (a horizontal state) is turned in a predetermined direction (arrow X3);

FIG. 17 is a plan view of the lower side illustrating a state wherein the treatment unit in the state shown in FIG. 14 (a horizontal state) is turned in a predetermined direction (arrow X3);

FIG. 18 is a side view illustrating a state wherein the treatment unit in the state shown in FIG. 12 (a horizontal state) is displaced to an opened state;

FIG. 19 is a side view illustrating a state wherein the treatment unit in the state shown in FIG. 18 (wherein the treatment unit is in a horizontal state and an opened state) is turned upwards;

FIG. 20 is a conceptual diagram of the link configuration, conceptually illustrating the link configuration of the surgical instrument according to the first embodiment of the present invention;

FIG. 21 is an enlarged perspective view of principal members illustrating around the treatment unit of a surgical instrument according to a second embodiment of the present invention, primarily showing the upper side of the treatment unit;

FIG. 22 is an enlarged perspective view of principal members, primarily showing the lower side of the treatment unit shown in FIG. 21;

FIG. 23 is an enlarged perspective view of principal members illustrating primarily the lower side in a state wherein a part of the members (the handle and the guard) have been removed from the operating unit of the surgical instrument according to the present embodiment;

FIG. 24 is a perspective view illustrating the general overall configuration of a surgical instrument according to a third embodiment of the present invention;

FIG. 25 is an enlarged perspective view of principal members illustrating around the treatment unit of the surgical instrument shown in FIG. 24, primarily showing the upper side of the treatment unit;

FIG. 26 is an enlarged perspective view of principal members illustrating around the treatment unit of the surgical instrument shown in FIG. 24, primarily showing the lower side of the treatment unit;

FIG. 27 is an enlarged perspective view of principal members with the treatment unit shown in FIG. 25 disposed in an opened state, primarily showing the upper side of the treatment unit;

FIG. 28 is an enlarged perspective view of principal members illustrating around the operating unit of the surgical instrument shown in FIG. 24, primarily showing the upper side of the operating unit;

FIG. 29 is an enlarged perspective view of principal members illustrating around the operating unit of the surgical instrument shown in FIG. 24, primarily showing the lower side of the operating unit;

FIG. 30 is an enlarged perspective view of principal members illustrating a state wherein a part of the members (the handle and a part of the guard) have been removed from the operating unit shown in FIG. 28;

FIG. 31 is an enlarged perspective view of principal members illustrating primarily the upper side in a state wherein a part of the members (the handle and the guard) have been removed from the operating unit shown in FIG. 28;

FIG. 32 is an enlarged perspective view of principal members illustrating primarily the lower side in a state wherein a part of the members (the handle and the guard) have been removed from the operating unit shown in FIG. 28;

FIG. 33 is a conceptual diagram of the link configuration, conceptually illustrating the link configuration of the surgical instrument according to a third embodiment of the present invention;

FIG. 34 is an enlarged perspective view of principal members illustrating around the treatment unit of a surgical instrument according to a fourth embodiment of the present invention, primarily showing the upper side of the treatment unit;

FIG. 35 is an enlarged perspective view of principal members illustrating around the treatment unit of a surgical instrument according to the present embodiment, primarily showing the lower side of the treatment unit;

FIG. 36 is an enlarged perspective view of principal members illustrating primarily the lower side in a state wherein a part of the members (the handle and the guard) have been removed from the operating unit of the surgical instrument according to the present embodiment; and

FIG. 37 is a conceptual diagram of the link configuration, conceptually illustrating the link configuration of the surgical instrument according to a fifth embodiment of the present invention.

### DETAILED DESCRIPTION OF THE PREFERRED EMBODIMENTS

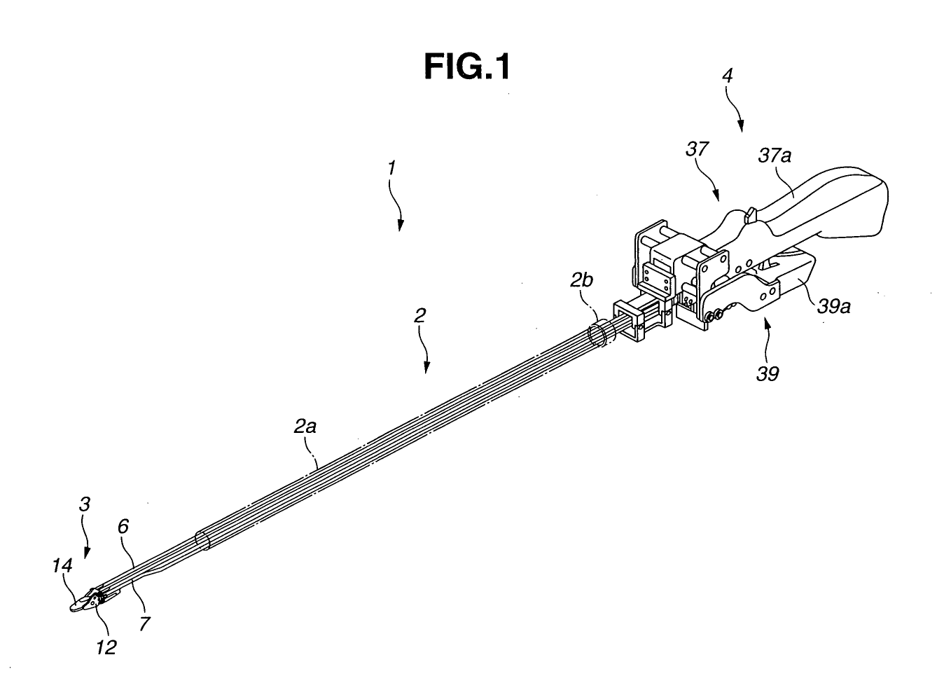

First, a schematic configuration of the surgical instrument according to the present embodiment will be described with reference to FIG. 1.

The surgical instrument 1 according to the present embodiment is an instrument used for performing surgical operations, and endoscopic surgical operations in particular, and is a multiple-degree-of-freedom forceps device configured so as to turn and open and close a treatment unit 3 provided on the tip portion of an insertion portion 2, by a surgeon performing predetermined operations while holding an operating unit 4 provided on the base portion of the insertion portion 2.

This surgical instrument 1 comprises the insertion portion 2 which is configured of multiple slender rods (6, 7, and so forth) and supporting members (8, 31) for the surgical instrument 1 and so forth, a treatment unit 3 configured of two treatment pieces (12, 14) provided on the tip side of this insertion portion 2, a linking mechanism for operating these two treatment pieces and the like, and an operating unit 4 formed of handles (37, 39) and the like configured of grips (37a, 39a) and the like provided on the base side of the above-described insertion portion 2 and a link mechanism and the like for transmitting force generated by operating the handles to the treatment unit 3, as shown in FIG. 1.

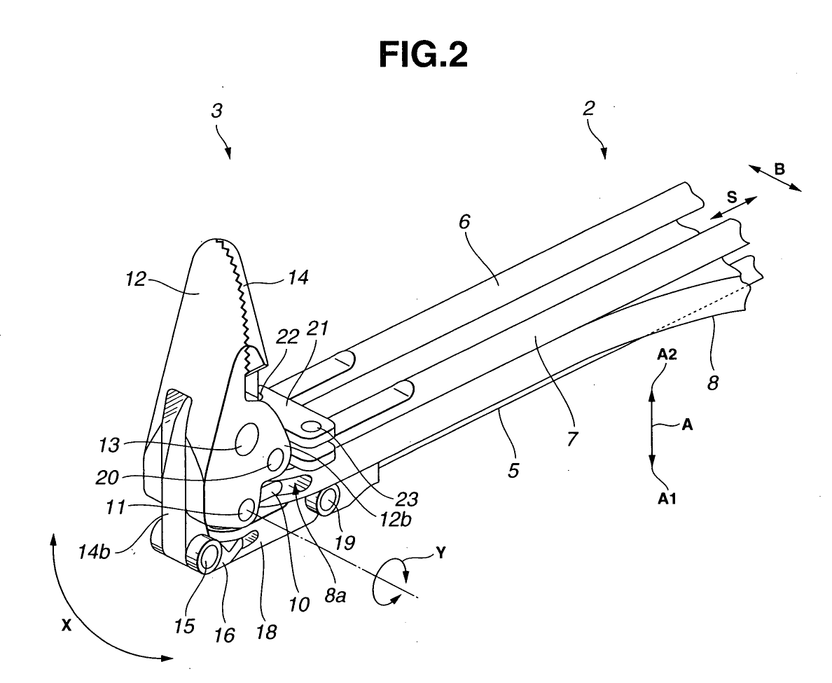

With the insertion portion 2, driving rods formed of multiple slender rods for mechanically connecting between the treatment unit 3 and the operating unit 4, i.e., a first driving rod 5 (Cannot be shown in FIG. 1. See FIG. 2 and subsequent drawings.), a second driving rod 6, and a third driving rod 7, are arrayed following the axial direction of the insertion portion 2 (the direction indicated by the arrow S in FIG. 2 through FIG. 4 and so forth).

Of these, the first driving rod 5 is a member making up a part of an opening/closing mechanism (opening/closing operating means) for realizing the opening/closing actions of the treatment unit 3, following predetermined operations of the operating unit 4. Also, the second driving rod 6 and the third driving rod 7 are disposed mutually in parallel or generally in parallel, serving as members making up a turning link for realizing the turning actions of the treatment unit 3, following predetermined operations of the operating unit 4.

The first driving rod 5 is disposed at a predetermined position offset to one side from the axial center of the insertion portion 2 (Toward the side below the present surgical instrument 1. The side in the direction of the arrow A1 in FIG. 2 through FIG. 4 and so forth.). Also, the second driving rod 6 and third driving rod 7 are respectively arrayed mutually in parallel or generally in parallel following the direction indicated by the arrow S in FIG. 2 through FIG. 4 and so forth as described above, and are respectively disposed at predetermined positions offset to the other side from the axial center of the insertion portion 2 (Toward the side above the present surgical instrument 1. The side in the direction of the arrow A2 in FIG. 2 through FIG. 4 and so forth.). The multiple driving rods 5, 6, 7) are each provided so as to be capable of advancing and retreating with regard to the axial direction S of the insertion portion 2.

Note that a sheath 2a, airtight means 2b (the members illustrated with dotted lines in FIG. 1), and so forth, are provided on the perimeter position of the insertion portion 2. The sheath 2a and airtight means 2b and other like members are components necessary for performing endoscopic surgical operations with a trocar (forceps hole), but are not directly related to the present invention, so description thereof in the present embodiment will be omitted, with the understanding that commonly-used sheaths, airtight means, and so forth will be used for a conventional surgical instrument.

Next, the detailed configuration of the treatment unit 3 and the operating unit 4 of the surgical instrument 1 according to the present embodiment will be respectively described below with reference to FIG. 2 through FIG. 11.

First, the detailed configuration of the treatment unit 3 of the surgical instrument 1 according to the present embodiment will be described primarily with reference to FIG. 2 through FIG. 4.

As described above, the treatment unit 3 is provided to the tip side of the insertion portion 2 as also illustrated in FIG. 2 through FIG. 4, and is configured of a first treatment piece 12 which is a turning treatment piece capable of turning as to a supporting member 8, a second treatment piece 14 which is an opening/closing treatment piece which is connected to the first treatment piece 12 through a first opening/closing pivot pin 13 which is a first opening/closing shaft so as to be able to be opened and closed, and a link mechanism and the like for causing the two treatment pieces (12, 14) to operate.

A first supporting portion 8 making up a part of the supporting member of the insertion portion 2, having predetermined rigidity and provided so as to protrude forwards (toward the treatment unit 3), is formed on the tip side of the insertion portion 2 (on the side toward the treatment unit 3). A slot portion 8a with a generally C-shaped cross-sectional form is formed at the tip portion of the first supporting portion 8. A first pivot shaft 9, having an axial center following the direction of the arrow A shown in the drawings, which is orthogonal to the axial direction S of the insertion portion 2, is positioned in this slot portion 8a, so as to pass therethrough.

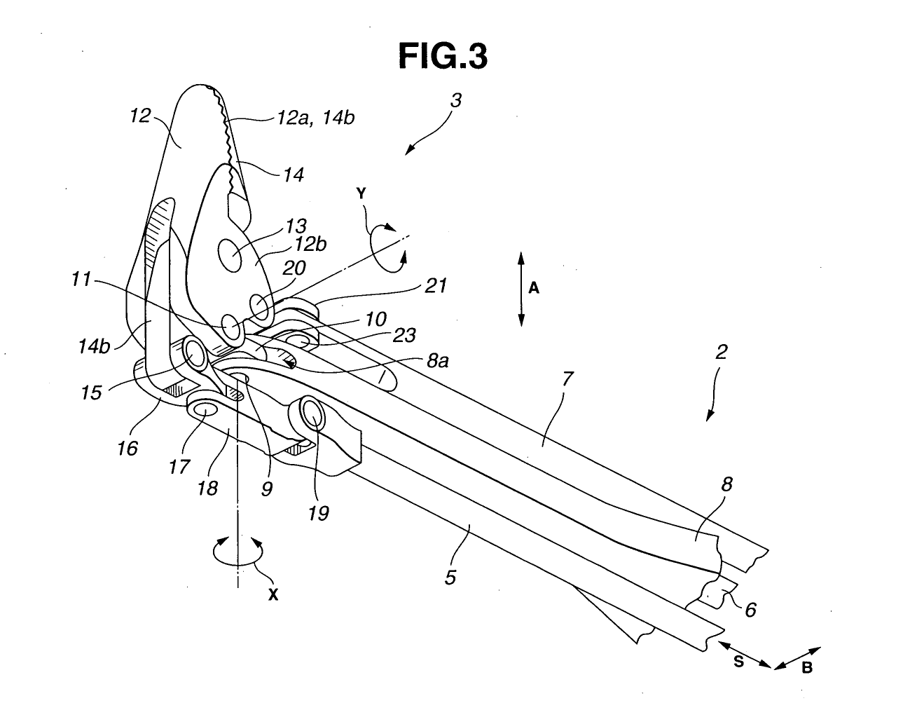

A first turning plate 10 has a predetermined member thereof is held within the slot portion 8a, and the first pivot shaft 9 passes through the first turning plate 10 as well as the slot portion 8a. Thus, the first supporting portion 8 and the first turning plate 10 are connected through the first pivot shaft 9, and also, the first turning plate 10 can turn on the first pivot shaft 9 in the direction of the arrow X shown in FIG. 2 and FIG. 3, and the like, as to the first supporting portion 8, within a predetermined range.

Also, a first pivot pin 11 which is a first turning shaft having an axial center in a direction orthogonal to the first pivot shaft 9 is provided on the other edge portion of the first turning plate 10. The first pivot pin 11 passes through a base portion 12b of the first treatment piece 12 at a predetermined position and is linked thereto.

That is to say, the other edge portion of the first turning plate 10 and the base portion 12b of the first treatment piece 12 are linked by the first pivot pin 11 so as to be capable of turning. Accordingly, in this case, the first treatment piece 12 can turn as to the first turning plate 10 being axially supported by the first pivot pin 11, in the direction of the arrow Y shown in FIG. 2 through FIG. 4 and so forth, within a predetermined range.

On the other hand, the second driving rod 6 and the third driving rod 7 are connected to the base portion 12b of the first treatment piece 12 through predetermined means near the first pivot pin 11 (details will be describe later).

Also, a first opening/closing pivot pin 13 having an axial center in the direction following the arrow B shown in the drawings, is fixed at a predetermined position partway along the first treatment piece 12. On the other hand, the base portion 12b of the first treatment piece 12 is formed in the shape of a slot, with a second treatment piece 14 being held in this slot-shaped portion partway. The first opening/closing pivot pin 13 passes through the second treatment piece 14 at a predetermined position. Thus, the second treatment piece 14 is configured so as to be able to turn in the same direction as the direction of the arrow Y shown in the drawings, axially supported by the first opening/closing pivot pin 13. That is to say, the second treatment piece 14 can open and close as to the first treatment piece 12.

Also, an arm portion 14b of the base side of the second treatment piece 14 has a first linking pin 15 having an axial center in the direction following the arrow B shown in the drawings, at the tip portion thereof. The first linking pin 15 serves as linking means for linking the first driving rod 5 for driving the opening and closing of the treatment unit 3, and the treatment unit 3, and links a first linking member 16 with the tip portion of the arm portion 14b of the base side of the second treatment piece 14 by passing through one end portion of the first linking member 16 making up a part of the opening/closing linking mechanism serving as opening/closing operating means of the treatment portion 3. Accordingly, the second treatment piece 14 is configured so as to be able to turn as to the first linking member 16 on the same direction as the arrow Y shown in the drawings, axially supported by the first linking pin 15.

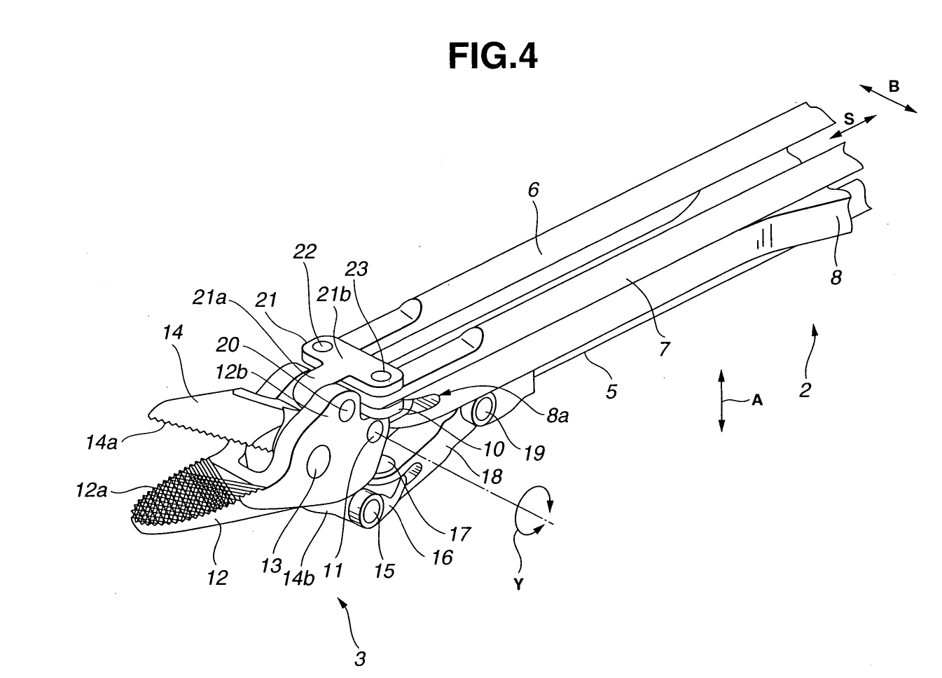

Further, a second linking pin 17 (Cannot be shown in FIG. 2. See FIG. 3 and FIG. 4) having an axial center in the direction following the arrow A shown in the drawings is provided at the other end portion of the first linking member 16, so as to pass therethrough. One end portion of a second linking member 18 making up the other part of the linking means for linking the first driving rod 5 for driving the opening and closing of the treatment unit 3, and the treatment unit 3, is connected to this second linking pin 17.

That is to say, one end portion of the second linking member 18 is formed in a generally slot-like shape, and disposed so that the other end portion of the first linking member 16 is held by this slot-shaped portion. The second linking pin 17 passes through the one end portion of the second linking member 18 and the other end portion of the first linking member 16 at a predetermined position, whereby the first linking member 16 is configured so as to be able to turn as to the second linking member 18 in the same direction as the X direction shown in the drawings, axially supported by the second linking pin 17.

Also, a third linking pin 19 having an axial center in the direction following the arrow B shown in the drawings is provided to the other end portion of the second linking member 18. The third linking pin 19 connects the other end portion of the second linking member 18 with the tip portion of the first driving rod 5 by passing through the tip portion of the first driving rod 5. Thus, the second linking member 18 is configured so as to be able to turn as to the first driving rod 15 in the same direction as the direction of the arrow Y shown in the drawings, axially supported by the third linking pin 19.

Thus, the opening/closing operating means (opening/closing link mechanism) on the treatment unit 3 side for operating the opening/closing actions of the treatment unit 3 is configured of the first linking member 16, the second liking pin 17 passing through the predetermined one end portion thereof, the second linking member 18 having the second linking pin 17 on the one end, the third linking pin 19 provided on the other end of the second linking member 18, the first driving rod 5, and so forth.

On the other hand, the second driving rod 6 and third driving rod 7 are connected to the base portion 12b of the first treatment piece 12 near the first pivot pin 11, by predetermined means, as described above.

That is to say, a second pivot pin 20 having an axial center in the direction following the arrow B shown in the drawings is provided on the base portion 12b of the first treatment piece 12 near the first pivot pin 11, and connected to this second pivot pin 20 is a protrusion 21a of a second turning plate 21 making up a part of the linking means for linking the second driving rod 6 and third driving rod 7 for driving the treatment unit 3 so as to turn, with the treatment unit 3.

The second turning plate 21 is a member formed of a main portion 21b having two broad plate members, and a protrusion 21a which protrudes outwards in one direction from a partway position of the main portion 21b so as to support the two plate members of the main portion 21b.

The second pivot pin 20 passes through the protrusion 21a in a direction following the arrow B shown in the drawing. Thus, the protrusion 21a of the second turning plate 21 is linked with a predetermined position on the base portion 12b of the first treatment piece 12, and the first treatment piece 12 is configured so as to turn as to the second turning plate 21 in the same direction as the arrow Y direction in the drawings within a predetermined range, axially supported by the second pivot pin 20.

A first turning pin 22 and a second turning pin 23 having axial centers in the directions following the arrow A in the drawings are provided to each end of the main portion 21b of the second turning plate 21 near both end portions in the direction following the arrow B in the drawings.

The second driving rod 6 for driving the turning of (the first treatment piece 12 of) the treatment unit 3 is linked to the first turning pin 22 at the one end portion, and the third driving rod 7 for driving the turning of (the first treatment piece 12 of) the treatment unit 3 is linked to the second turning pin 23 at the other end portion, in the same way.

Accordingly, the configuration is such that mutually advancing and retreating the second driving rod 6 and third driving rod 7 in the direction following the arrow S in the drawings, according to the predetermined operations of the operating unit 4, causes the second turning plate 21 to be capable of turning in the direction of the arrow X shown in FIG. 3, within a predetermined range.

Also, gripping faces 12a and 14a are formed on each predetermined position of the first treatment piece 12 and second treatment piece 14 which are the principal members making up the treatment unit 3. Predetermined recesses and protrusions are formed on the gripping faces 12a and 14a as necessary, so as to allow objects to be held, such as stitching needles, stitching thread, body tissue, etc., in a sure manner. The gripping faces 12a and 14a are disposed so as to face one another in the state that both treatment pieces 12 and 14 are assembled, as shown in FIG. 4.

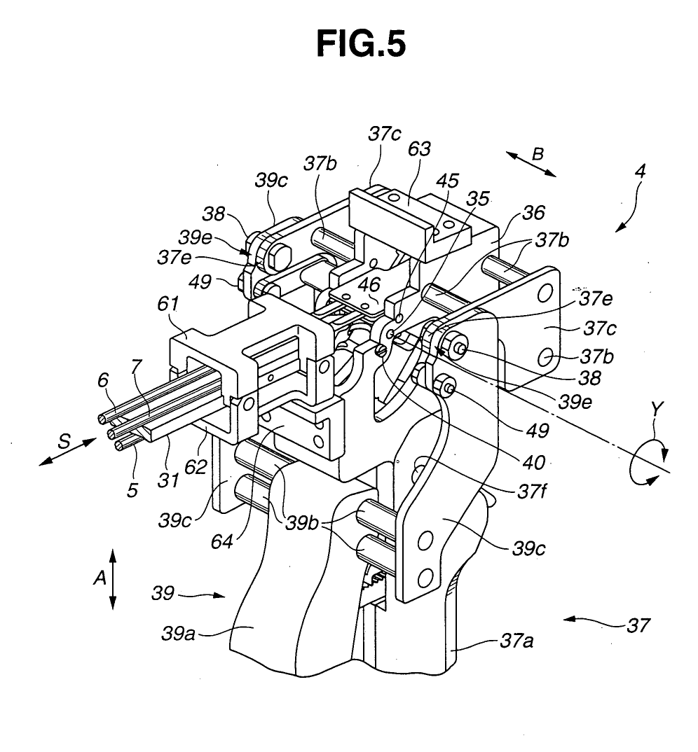

Next, the detailed configuration of the operating unit 4 of the surgical instrument 1 according to the present embodiment will be described primarily with reference to FIG. 5 through FIG. 11.

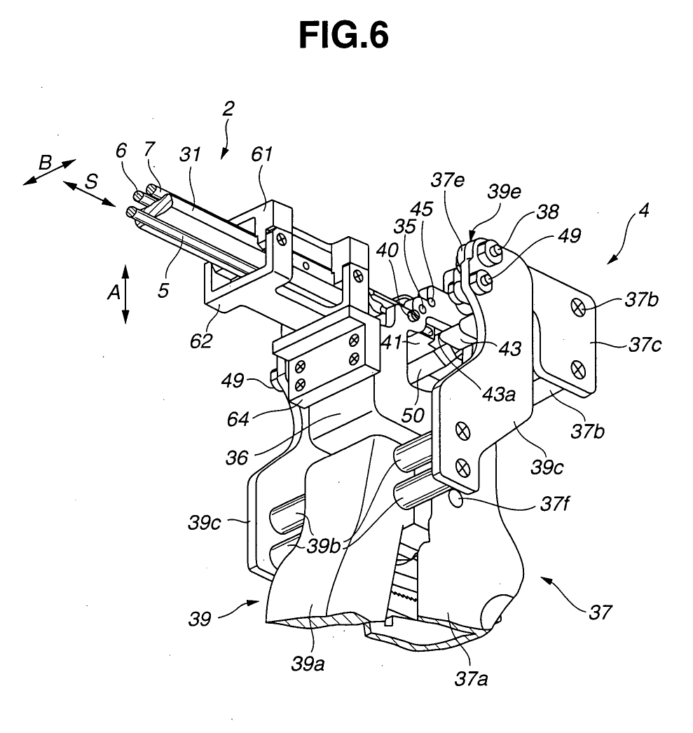

As described above, the operating unit 4 is provided on the base side of the insertion portion 2 as also illustrated in FIG. 5 and FIG. 6, and is configured of a first handle 37 which is a turning operating piece capable of turning as to the supporting member 31, a second handle 39 which is an opening/closing operating piece connected to the first handle 37 through a second opening/closing shaft (38) so as to be capable of being opened and closed, a linking mechanism for transmitting force generated by operating these two handles (37, 39) to the treatment unit 3, an so forth.

The first handle 37 comprises the components of a first grip 37a which is a member to be held by the surgeon, a side supporting plate 37c for supporting the side of the operating unit 4, a first handle linking member 36 for linking the operating unit 4 to the insertion portion 2, and so forth. Also, the second handle 39 comprises the components of a second grip 39a which is a member to be held by the surgeon, a second handle linking member 39c for linking the second grip 39a to the first handle 37, and so forth.

Of the members making up the first handle 37, the first grip 37a is formed so as to be an optimal shape for the surgeon to hold using the thumb, fourth finger, and little finger, when operating the surgical instrument 1. Also, of the members making up the second handle 39, the second grip 39a is formed so as to be an optimal shape for the surgeon to operate using the index finger and middle finger, when operating the surgical instrument 1.

Support shafts 37b formed of multiple shaft-like members with an axial center following the direction of the arrow B in FIG. 5 and FIG. 6 are erected on both side faces of the first handle linking member 36 provided on one end side of the first handle 37, facing outwards. Two side face supporting plates 37c formed of plate-shaped members having generally L-shaped flat faces are integrally disposed on the tip portions of the support shafts 37b, so as to follow planes orthogonal to the support shafts 37b. That is to say, the side face supporting plates 37c are respectively disposed with a predetermined distance (the length of the support shafts 37b) from both side portions of the one end side of the first handle linking member 36.

Second opening/closing pivot pins 38 having axial centers in the direction following the arrow B shown in the drawings are each erected near the tip portion of the arm portions 37e of the side face supporting plates 37c. The second opening/closing pivot pins 38 are integrally axially supported at a predetermined position near the one end portions 39e of the second handle linking member 39c of the second handle 39. Thus, the second handle 39 is configured so as to be capable of turning as to the first handle 37 in the same direction as the direction of the arrow Y shown in the drawings, axially supported by the second opening/closing pivot pins 38.

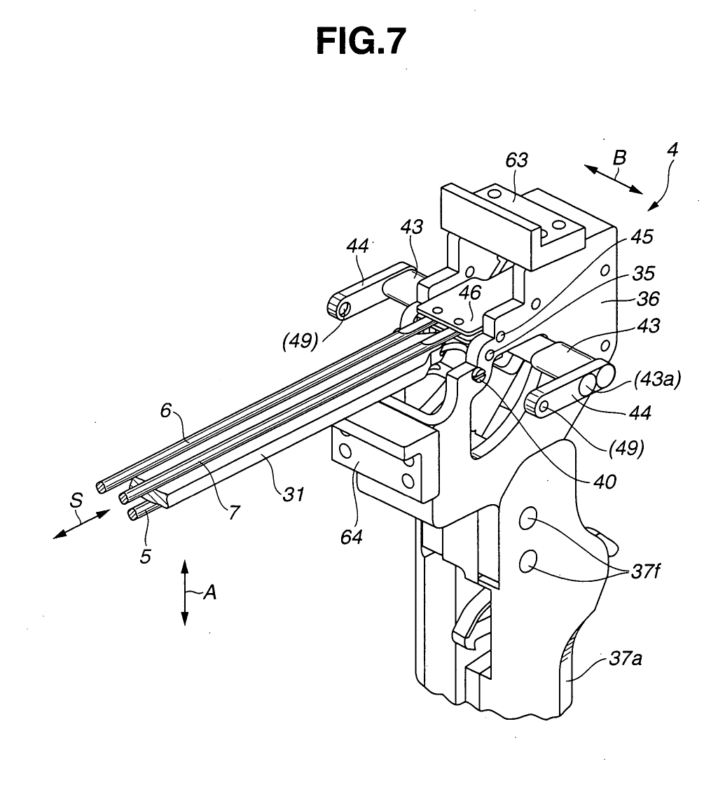

Also, fifth linking pins 49 are disposed near the second opening/closing pivot pins 38, having axial centers following the same direction so as to be generally parallel thereto. The free end portions of fifth linking members 44 having lever-like shapes as shown in FIG. 7, are linked to the fifth linking pins 49 so as to be capable of turning.

Fourth linking members 43 having axial centers in the direction following the arrow B shown in the drawings are fixed at the base portion of the fifth linking members 44. Point-of-force shafts 43a having axial centers in the direction following the arrow B in the drawings are disposed at a position partway along the fourth linking members 43. Further, the point-of-force shafts 43a fit into point-of-force shaft receiving holes 41c disposed at predetermined positions on a later-described third linking member 41, so as to be capable of turning.

Multiple second handle linking shafts 39b having axial centers in the direction following the arrow B shown in the drawings are erected in the inwards direction near the other end portion of the second handle linking member 39c. The end portions of the multiple second handle linking shafts 39b are each fixed to predetermined parts on both side faces of the second grip 39a toward one side. Thus, the second grip 39a and the second handle linking member 39c are formed integrally.

Also, the first handle linking member 36 and the first grip 37a are connected by a first handle linking shaft 37f as shown in FIG. 7, thereby integrally forming the two.

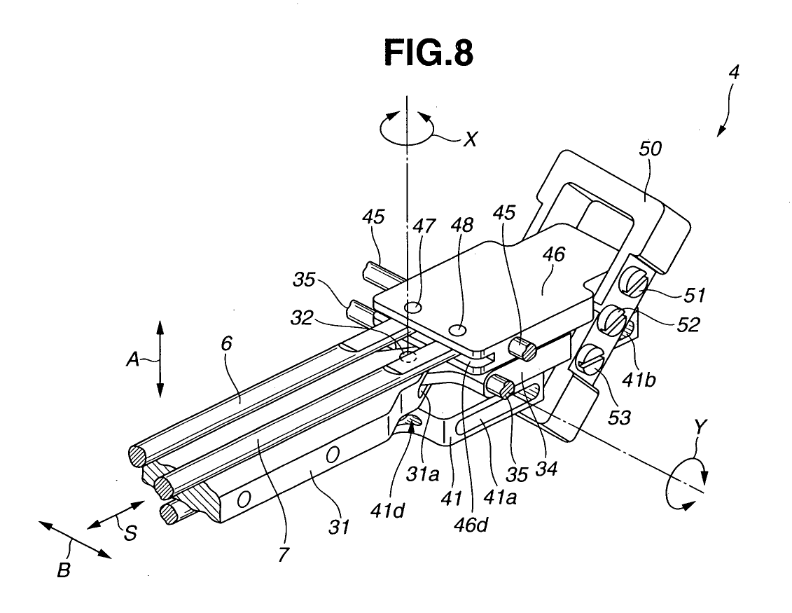

On the other hand, a second supporting portion 31 making up a part of the supporting member of the insertion portion 2, having predetermined rigidity and protruding backwards (toward the operating unit 4), is provided on the base side (the side toward the operating unit 4) of the insertion portion 2. The tip portion of the second supporting portion 31 has a slot portion 31a having a generally C-shaped cross-section formed, as shown in FIG. 8 through FIG. 11. A second pivot shaft 32 having an axial center in the direction following the arrow A shown in the drawings, which is the direction orthogonal to the axial direction S of the insertion portion 2, so as to pass therethrough, is disposed on the slot portion 31a.

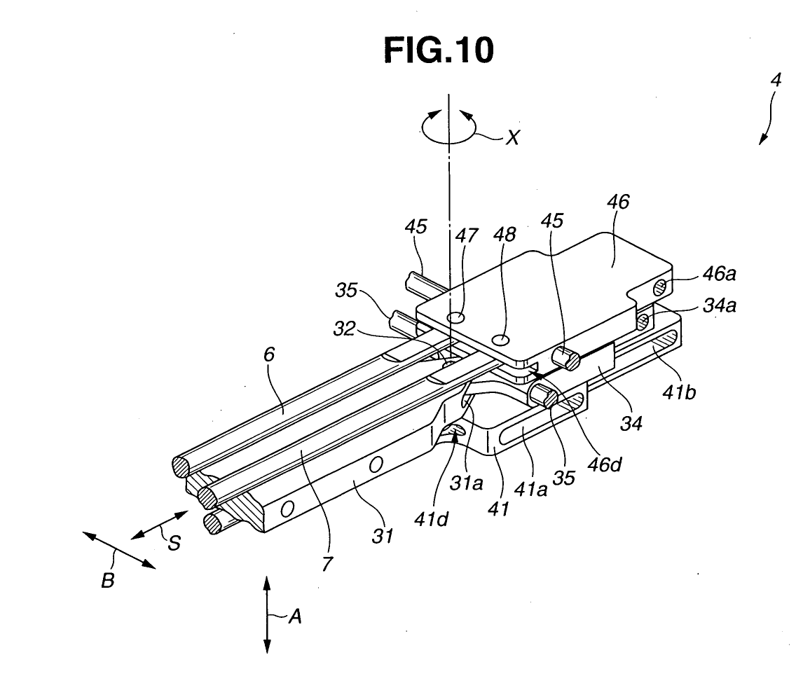

A predetermined part of one end side of a third turning plate 34 is held within the slot portion 31a, with the second pivot shaft 32 passing through both the first turning plate 10 and the slot portion 31a. Thus, the second supporting portion 31 and the third turning plate 34 are linked through the second pivot shaft 32, and also, the third turning plate 34 is configured so as to be capable of turning as to the second supporting portion 31 in the direction of the arrow X shown in FIG. 8 and FIG. 10 and so forth, within a predetermined range, axially supported by the second pivot shaft 32.

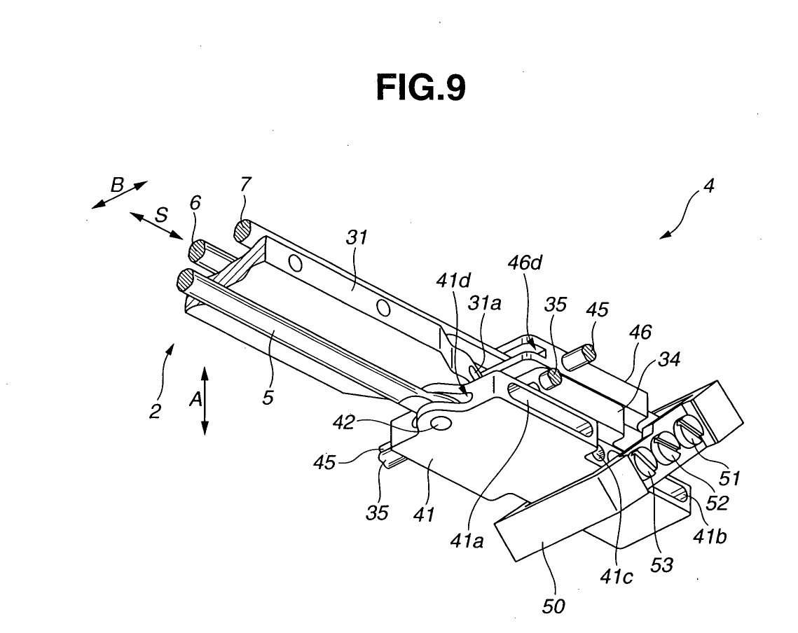

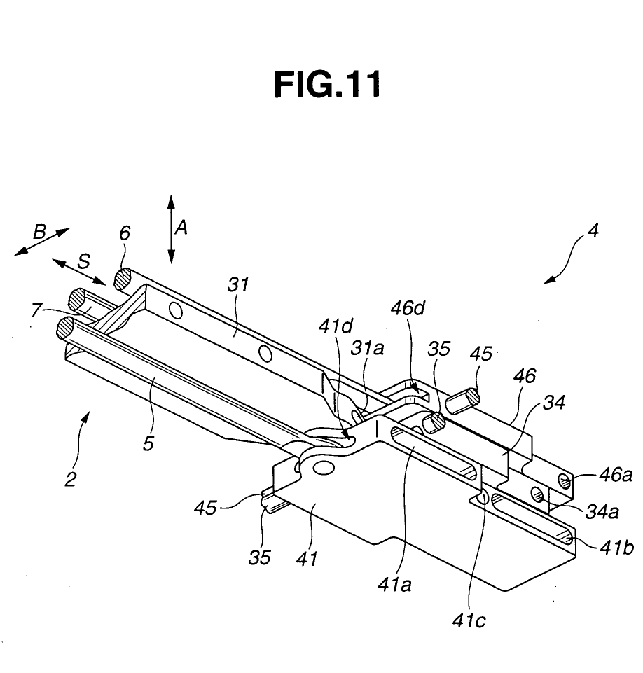

A second parallel guide pin receiving hole 34a which is a through hole having an axial center in the direction following the arrow B shown in the drawings, as shown in FIG. 10 and FIG. 11, is bored at a predetermined portion at the other end side of the third turning plate 34. A later-described second parallel guide pin 52 is stored within this second parallel guide pin receiving hole 34a so as to be capable of turning (see FIG. 8 and FIG. 9).

Also, a third pivot pin 35 having an axial center in the direction following the arrow B shown in the drawings, as shown in FIG. 8 through FIG. 11, is disposed at a predetermined position on the side face of the third turning plate 34 and toward one end thereof. A predetermined portion of the first handle 37 is linked to this third pivot pin 35 as shown in FIG. 5 through FIG. 7. Thus, the first handle 37 is configured so as to be capable of turning in the direction of the arrow Y shown in FIG. 5, FIG. 8, and so forth, within a predetermined range, axially supported by the third pivot pin 35.

On the other hand, of the multiple driving rods (5, 6, 7), the first driving rod 5 is linked to the third linking member 41 through later-described predetermined means, and the second driving rod 6 and the third driving rod 7 are linked to the fourth turning plate 46 through later-described predetermined means.

That is to say, the second driving rod 6 and the third driving rod 7 have the end portions thereof each formed in the general shape of a flat plate, as shown in FIG. 8 and FIG. 10, with these end portions fitting into a slot portion 46d formed on one end of the fourth turning plate 46. Two turning pins having axial centers in the direction following the arrow A shown in the drawing so as to pass through the slot portion 46d of the fourth turning plate 46, i.e., a third turning pin 47 and a fourth turning pin 48, are provided. Each of the third turning pin 47 and the fourth turning pin 48 pass through each of the second driving rod 6 and the third driving rod 7 along with the slot portion 46d.

Thus, in the event that the fourth turning plate 46 turns in the same direction as the direction of the arrow X shown in FIG. 8, FIG. 10, and so forth, along with a predetermined operation of the operating unit 4, within a predetermined range, the second driving rod 6 and the third driving rod 7 follow this movement so as to mutually advance and retreat in the direction following the arrow S shown in the drawings.

Also, a first parallel guide pin receiving hole 46a which is a through hole having an axial center in the direction following the arrow B shown in the drawings, as shown in FIG. 10 and FIG. 11, is bored at a predetermined portion at the other end side of the fourth turning plate 46. The first parallel guide pin receiving hole 46a has a later-described fist parallel guide pin 51 stored therein so as to be capable of turning (see FIG. 8 and FIG. 9).

Further, fourth pivot pins 45 having an axial center in the direction following the arrow B shown in the drawings, as shown in FIG. 8 through FIG. 11, is disposed at a predetermined position on both side faces of the fourth turning plate 46 close to the slot portion 46d. A predetermined portion of the first handle 37 is linked to the fourth pivot pin 45 as shown in FIG. 5 through FIG. 7. Accordingly, the tip side of the fourth turning plate 46 is capable of turning in the same direction as the direction of the arrow Y shown in FIG. 5, FIG. 8, and so forth, on the fourth pivot pin 45 within a predetermined range, axially supported by the fourth pivot pin 45, along with a predetermined operation of the operating unit 4 (first handle 37).

Also, the first driving rod 5 has the end portion thereof formed in the shape of a generally flat plate, as shown in FIG. 9 and FIG. 11, with this end portion fitting into the slot portion 41d formed at the one end portion of the third linking member 41. A fourth linking pin 42 having an axial center in the direction following the arrow A shown in the drawings is provided so as to pass through the slot portion 41d of the third linking member 41. This fourth linking pin 42 passes through the first driving rod 5 and the slot portion 41d together, thereby linking the first driving rod 5 with the third linking member 41.

In this way, the first driving rod 5, the third linking member 41, the point-of-force shafts 43a, the fourth linking pin 42, the fourth linking member 43, the fifth linking member 44, the fifth linking pin 49, and so forth, configure the opening/closing operating means (opening/closing link mechanism) of the operating unit 4 side which performs the opening/closing actions of the treatment unit 3.

On both side faces of the third linking member 41 are formed a first groove portion 41a having a long side in the direction following the arrow S in the drawings at a predetermined position toward one end (toward the slot portion 41d), a second groove portion 41b having a long side in the direction following the arrow S in the drawings at a predetermined position toward the other end, and point-of-force shaft receiving holes 41c having axial centers in the direction following the arrow B shown in the drawings at a predetermined middle position.

Guide pins 40 (see FIG. 5 through FIG. 7) provided on the first handle 37 near the third pivot pin 35 are slidably engaged with the first groove portions 41a. Also, later-described third parallel guide pins 53 slidably fit with the second groove portions 41b. The point-of-force shafts 43a of the above-described fourth linking member 43 (see FIG. 6 and FIG. 7) fit to the point-of-force shaft receiving holes 41c so as to be capable of turning.

Thus providing the first groove portions 41a and second groove portions 41b to the both side faces of the third linking member 41, and slidably fitting the guide pins 40 and the third parallel guide pins 53 (described later) to each, allows the third linking member 41 to advance and retreat in the forward and backward directions (in the direction of the arrow S) while maintaining the parallel relation with the third turning plate 34 and the fourth turning plate 46. Also, even in the event that the force applied to the point-of-force shaft receiving hole 41c of the third linking member 41 is directed away form the direction of the horizontal axis of the third linking member 41, the horizontal state of the third linking member 41 is not lost. Accordingly, the configuration is such that the parallel mapping relation between the third linking pin 19 of the treatment unit 3 and the corresponding point-of-force shaft 43a of the operating unit 4 can be maintained in a sure manner.

As described above, the first parallel guide pins 51, second parallel guide pins 52, and third parallel guide pins 53, are disposed at the other end portions of the fourth turning plate 46, third turning plate 34, and fourth linking member 43, with predetermined means. A cradle 50 formed in the shape of a basket, as shown in FIG. 8 and FIG. 9, is introduced between the fourth turning plate 46, third turning plate 34, and fourth linking member 43, and the parallel guide pins 51, 52, and 53. Accordingly, the fourth turning plate 46, third turning plate 34, and fourth linking member 43 are linked integrally with each other by the cradle 50.

Further, the first parallel guide pins 51, second parallel guide pins 52, and third parallel guide pins 53, are disposed at positions distanced from the fourth pivot pin 45 and third pivot pin 35 linked to the first handle 37 and the guide pin 40 provided to the first handle 37, by a generally equal distance, in the direction following the arrow S shown in the drawings. Accordingly, the fourth turning plate 46, third turning plate 34, and the third linking member 41 can be restricted to a constantly parallel relation within the range where the first handle 37 is capable of turning.

Due to such a configuration, the operating unit 4 of the surgical instrument 1 according to the present embodiment is linked to the second supporting portion 31 at the base side of the insertion portion 2, and the first handle 37 and the second handle 39 making up the operating unit 4 are configured so as to be capable of turning as to the second supporting portion 31 in the direction of the arrow X and the direction of the arrow Y shown in FIG. 8 and the like, axially supported by the second pivot shaft 32 and the third pivot pin 35.

Note that predetermined ratchet means and release spring means are provided between the first grip 37a and the second grip 39a making up a part of the operating unit 4. The ratchet means and release spring means are mechanisms for restricting the opening/closing state between the first grip 37a and the second grip 39a at a predetermined position or pressing the first grip 37a and the second grip 39a in the direction so as to open, but are not directly related to the present invention. Accordingly, description regarding the configuration of the ratchet means and the like will be omitted, with the understanding that conventional commonly-used articles will be used.

Also, protecting members such as a first guard member 61, a second guard member 62 for covering and protecting near a linking portion with the base portion of the inserting portion 2 for example, a third guard member 63 and a fourth guard member 64, for covering and protecting a predetermined portion of the first handle linking member 36, and the like, are provided to the operating unit 4, but these also are portions not directly related to the essence of the present invention, so description thereof will be omitted.

The operations of the surgical instrument 1 according to the present embodiment thus configured will be described next.

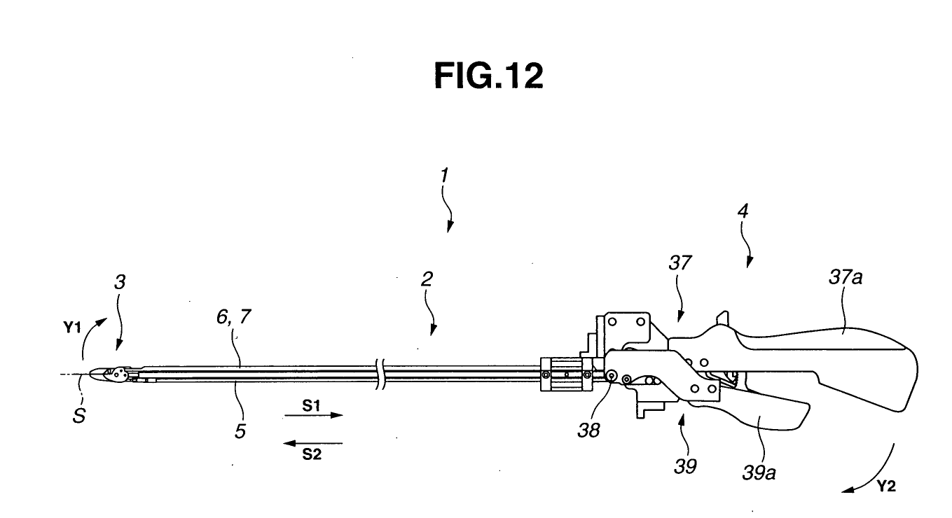

First, in the event of being in the state shown in FIG. 12, i.e., with the treatment unit 3 and the operating unit 4 both in a parallel state (a state wherein the angle of the insertion portion 2 as to the axial direction S is 0 degrees), the surgeon operating the surgical instrument 1 holds the predetermined position of the operating unit 4. Specifically, the surgeon holds the first grip 37a of the first handle 37 and the second grip 39a of the second handle 39.

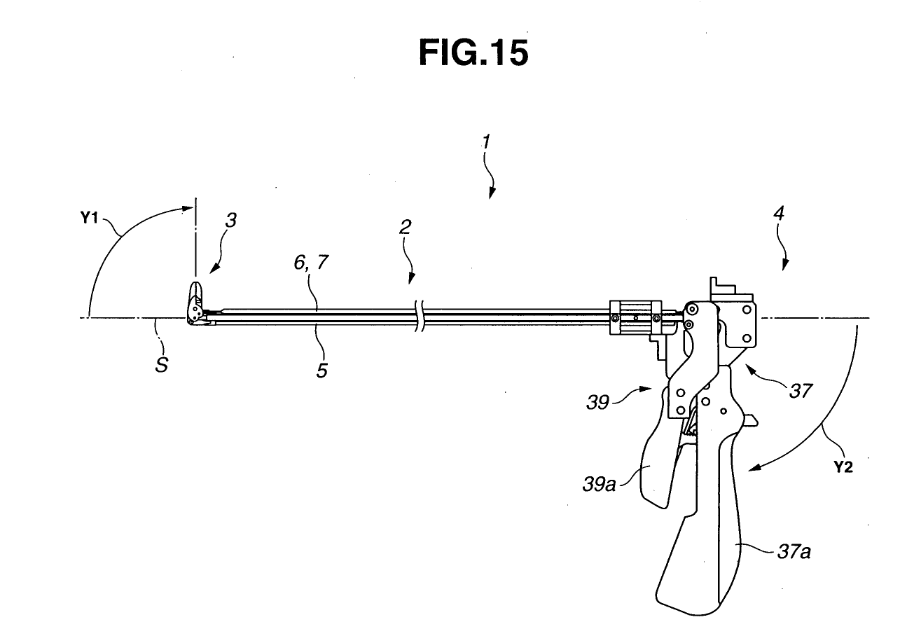

In this state, the first handle 37 is turned in the direction of the arrow Y2 shown in FIG. 12, centered on the third pivot pin 35. Accordingly, the second driving rod 6 and the third driving rod 7 move in the direction of the arrow S1 shown in FIG. 12 at the same time. Along with this, the first driving rod 5 moves in the direction of the arrow S2 shown in the drawing. As a result, the treatment unit 3 turns in the direction of the arrow Y1 shown in FIG. 12 with the first treatment piece 12 and the second treatment piece 14 configuring itself still closed. This results in the state shown in FIG. 15. That is, the treatment unit 3 turns toward the direction of the arrow Y1 (upwards) by a general angle of 90 degrees, and the operating unit 4 turns toward the direction of the arrow Y2 (downwards) by the same angle.

Accordingly, the treatment unit 3 and the operating unit 4 are capable of turning within the range between the state shown in FIG. 12 and the state shown in FIG. 15, with regard to the turning in the direction following the arrows Y1 and Y2 in FIG. 12 and FIG. 15.

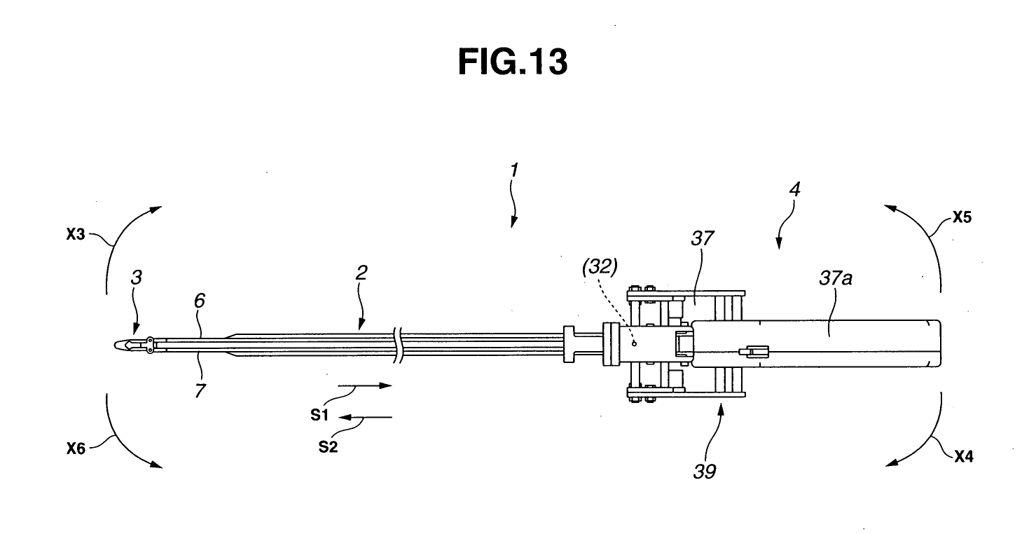

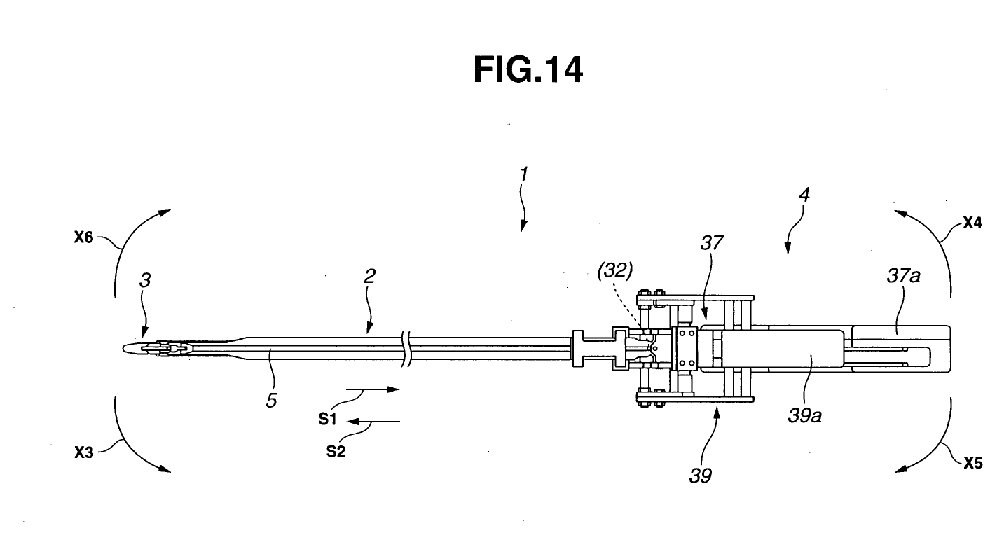

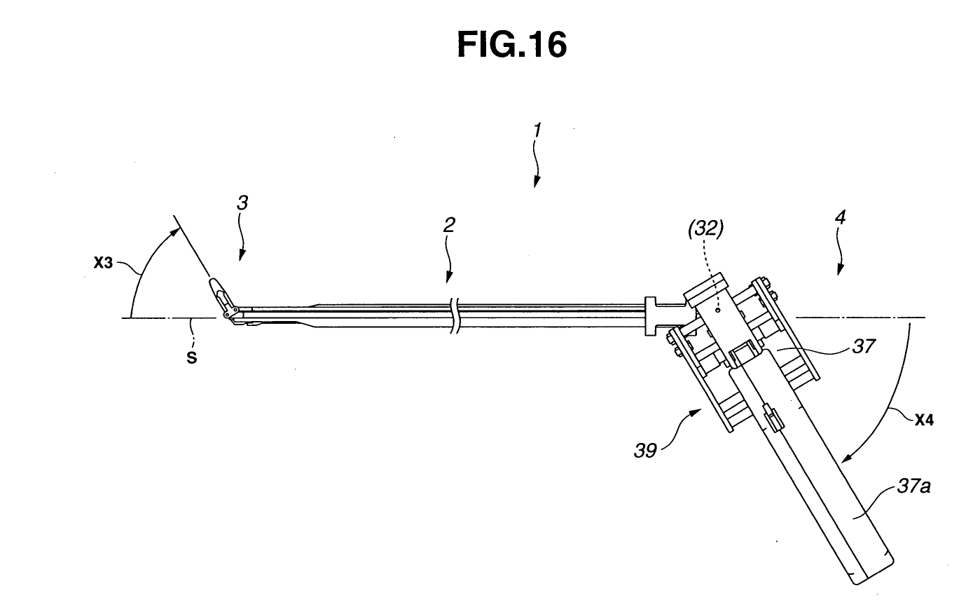

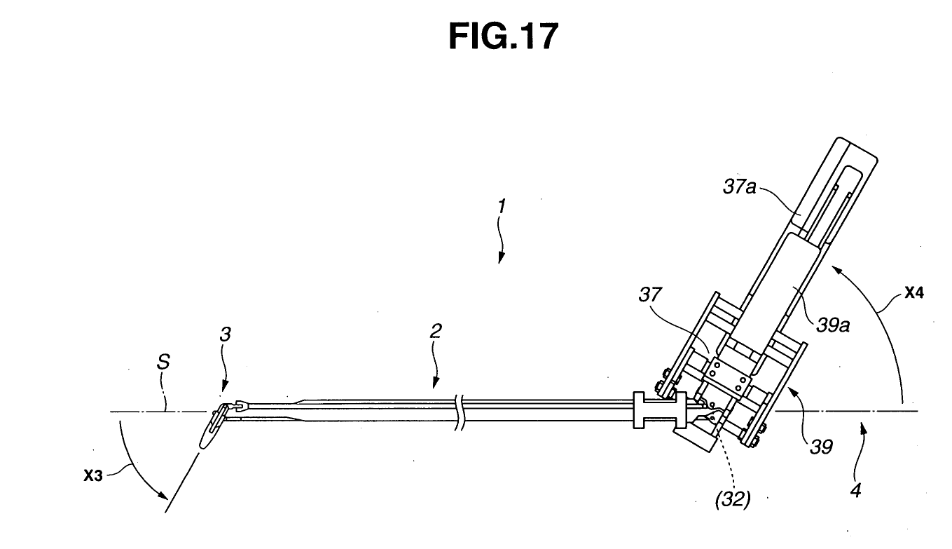

On the other hand, in the event of the state shown in FIG. 12 through FIG. 14 (in the state that the treatment unit 3 and the operating unit 4 are both in a horizontal state (a state wherein the angle of the insertion portion 2 as to the axial direction S is 0 degrees), the surgeon holds a predetermined position of the operating unit 4 (the first grip 37a of the first handle 37 and the second grip 39a of the second handle 39), and turns the operating unit 4 in the direction of the arrow X4 shown in FIG. 13 and FIG. 14. centered on the second pivot shaft 32. This causes one of the second driving rod 6 and the third driving rod 7 to advance and retreat in the direction of the arrow S1 shown in FIG. 12 through FIG. 14, and the other in the direction of the arrow S2 shown in the same drawings. At this time, the fourth linking pin 42 of the end portion of the first driving rod 5 at the side of the operating unit 4 is near the second pivot shaft 32 which is the center axis of turning of the operating unit 4, so there is hardly any advancing or retreating thereof. Consequently, the treatment unit 3 turns in the direction of the arrow X3 shown in FIG. 13 and FIG. 14 with the first treatment piece 12 and the second treatment piece 14 making up itself remaining in the closed state. This brings about the state shown in FIG. 16 and FIG. 17. That is to say, the state wherein the treatment unit 3 has turned in the direction of the arrow X3 by an angle of generally 60 degrees, and the operating unit 4 has also turned in the direction of the arrow X4 by the same angle.

Also, in the event of turning the operating unit 4 in the direction of the arrow X5 which is the opposite direction as to the arrow X4 in FIG. 13 and FIG. 14, the treatment unit 3 moves in the direction of the arrow X6 shown in the drawings by the same angle.

Accordingly, with regard to turning in the direction following the arrows X3 and X4 in FIG. 13 and FIG. 14, and FIG. 16 and FIG. 17, the treatment unit 3 and the operating unit 4 are capable of turning within the range between the state shown in FIG. 13 and FIG. 14, and the state shown in FIG. 16 and FIG. 17. In the same way, turning in the direction following the arrows X6 and X5 can be made within a turning range with the same angle.

With the surgical instrument 1 according to the present embodiment, turning of the treatment unit 3 in the vertical direction (in the direction of the arrow Y1 in FIG. 12) and turning in the horizontal direction (in the direction of the arrows S3 and X6 in FIG. 13 and FIG. 14) can be performed at the same time. That is to say, performing arbitrary turning operations of the operating unit 4 in the direction of the arrow Y2 in FIG. 12 and in the direction of the arrows S4 and X5 in FIG. 13 and FIG. 14 allows the treatment unit 3 to be arbitrarily turned within the predetermined range.

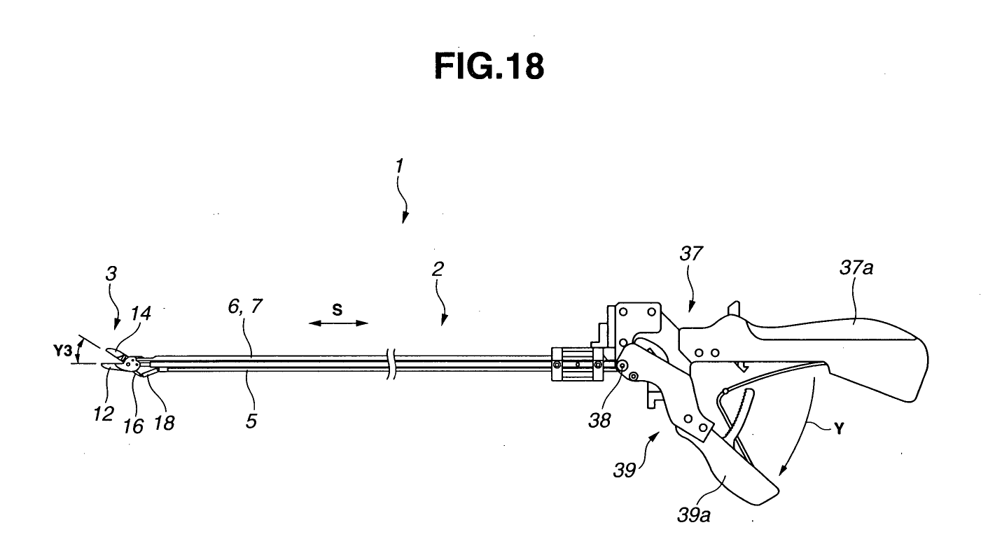

Further, in a state wherein the operating unit 4 has been arbitrarily turned, in the event that the surgeon operates the second handle 39 as to the first handle 37 in the direction indicated by the arrow Y shown in FIG. 18 (toward the opening direction), the second handle 39 turns within the predetermined range with the second opening/closing pivot pin 38 as the center of turning. Accordingly, the third linking member 41 advances and retreats in the direction of the arrow S shown in FIG. 18, along with the turning of the second handle 39 through the fifth linking member 44 and the fourth linking member 43 (see FIG. 6 and FIG. 7). At the same time, the first driving rod 5 also moves so as to advance and retreat in the same direction S along therewith.

Upon the first driving rod 5 advancing and retreating in this direction, the second linking member 18 and the first linking member 16 (see FIG. 4) also moves in the same direction, accordingly, the first treatment piece 12 turns the second treatment piece 14 in the direction of the arrow Y3 shown in FIG. 18, with the first opening/closing pivot pin 13 as the center or turning. Consequently, the second treatment piece 14 performs arbitrary opening/closing actions within a predetermined range, with regard to the first treatment piece 12.

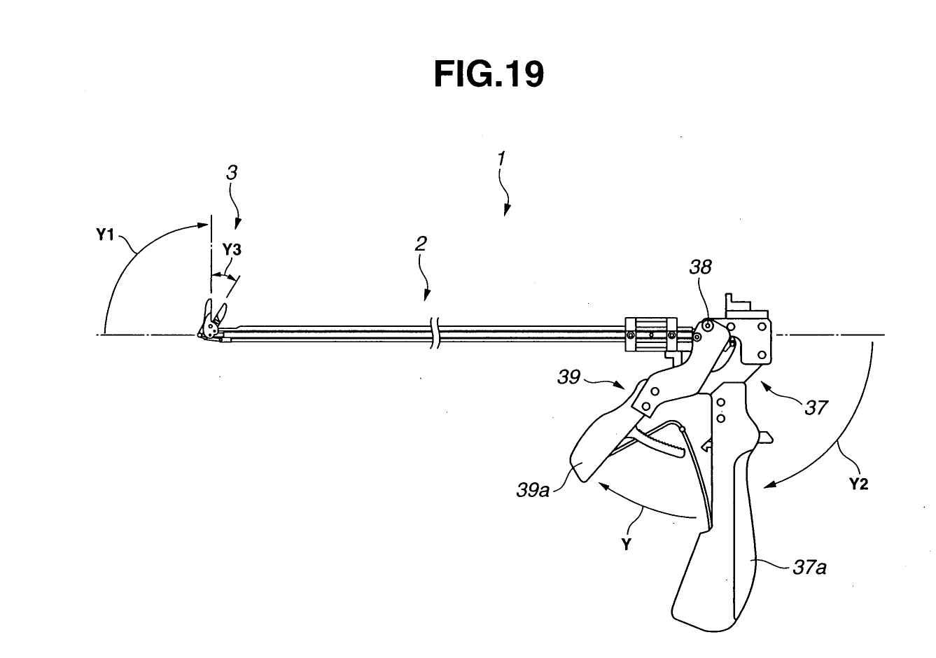

In the event that the operating unit 4 is turned in the direction of the arrow Y2 shown in FIG. 19 for example, with the treatment unit 3 in the opened state shown in FIG. 18, the treatment unit 3 turns in the direction of the arrow Y1 shown in the drawing with the opened state maintained. That is to say, the treatment unit 3 can be arbitrarily turned by performing turning operations of the operating unit 4 with the opened/closed state maintained. Also, in the event that the treatment unit 3 is in an arbitrary position within the movable range thereof, performing opening/closing operations of the operating unit 4 enables opening/closing actions of the treatment unit 3 to be executed at all times.

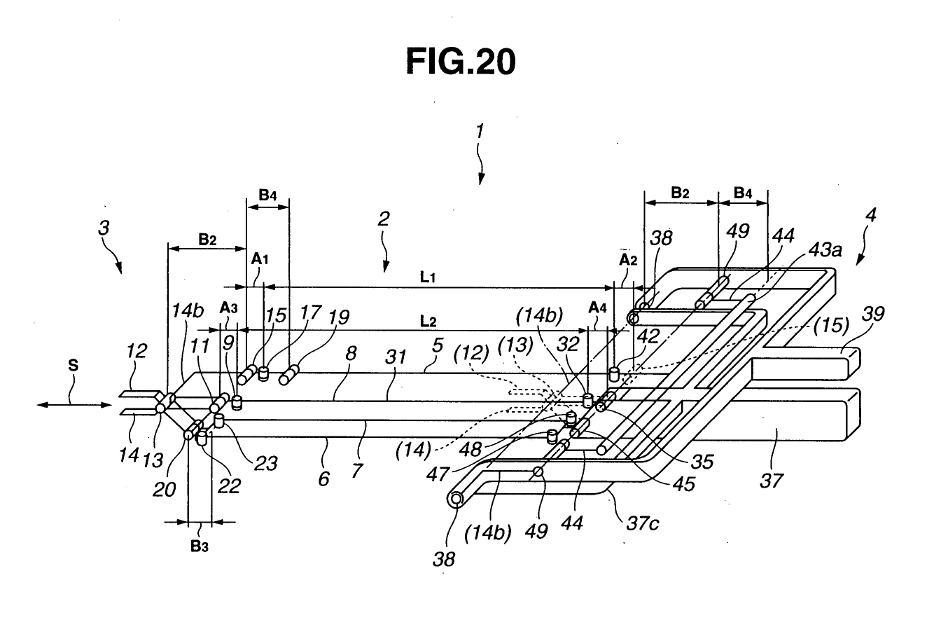

The disposedness of links for linking the components of the surgical instrument 1 according to the present embodiment described above, and the distance between the fulcrums (pins) and the shafts making this up, will be described with reference to the conceptual link configuration diagram in FIG. 20.

Note that what is shown in FIG. 20 with the dotted lines are imaginary lines of a case wherein the treatment unit 3 disposed at the tip side of the insertion portion 2 is moved toward the base side of the insertion portion 2, in parallel along the axial direction S. That is to say, this illustrates imaginary lines assuming eliminating the insertion portion 2 and directly linking the treatment unit 3 and the operating unit 4.

First, the link configuration of the treatment unit 3 is as follows. That is to say, as shown in FIG. 20, the first treatment piece 12 and the second treatment piece 14 are linked by the first opening/closing pivot pin 13 so as to be capable of opening and closing. The first pivot pin 11 (first turning shaft) and the second pivot pin 20 are linked at the base side of the first treatment piece 12, and the fist linking pin 15 (first joint) is linked to the base side of the second treatment piece 14 through the arm portion 14b of the second treatment piece 14 with a distance B2 therebetween.

At the base side of the first pivot pin 11, the first pivot shaft 9 (second turning shaft) is provided across a distance A3 in the direction of the arrow S shown in FIG. 20, and the first turning pin 22 and second turning pin 23 are provided at the base side of the second pivot pin 20 across the predetermined distance B3 (preferably set to the same distance as the distance A3)in the direction of the arrow S shown in FIG. 20.

Also, the second linking pin 17 (second joint) is provided at the base side of the first linking pin 15 across a distance A1 in the direction of the arrow S in this drawing, and further, the third linking pin 19 is provided at the base side of the second linking pin 17.

On the other hand, the link configuration of the operating unit 4 is as follows. That is, as shown in this drawing, the first handle 37 and the second handle 39 are linked with the second opening/closing pivot pin 38 so as to be capable of opening and closing. The third pivot pin 35 and fourth pivot pin 45 are linked at one end side of the first handle 37, and the fifth linking pin 49 (third joint) is linked at the one end side of the second handle 39, at a position across the same distance (distance B2) as the arm 14b of the second treatment piece 14.

The second pivot shaft 32 (turning shaft) is provided toward the treatment unit 3 (toward the front) from the third pivot pin 35 at a position across a distance A4, and the third turning pin 47 and the fourth turning pin 48 are provided in front of the fourth pivot pin 45 with a predetermined distance in the direction of the arrow S in the drawing (preferably set to the same distance as with the distance A4).

Also, the point-of-force shaft 43a is provided at one end side of the fifth linking pin 49 at a position across the same distance B4 as the distance between the first linking pin 15 and the third linking pin 19, and also, the fourth linking pin 42 (fourth joint) is provided at the other end side, across a predetermined distance.

Now, the shaft (pin) indicated by the imaginary reference numeral (15) on the dotted line exists at a position equivalent to that of the fifth linking pin 49. The fourth linking pin 42 is provided at a position on the one side of the fifth linking pin 49 across a distance A2 in the direction of the arrow S in the drawing.

That is to say, the first opening/closing pivot pin 13 is equivalent to the second opening/closing pivot pin 38, the first pivot pin 11 is equivalent to the third pivot pin 35, the second pivot pin 20 is equivalent to the fourth pivot pin 45, the first linking pin 15 is equivalent to the fifth linking pin 49, and the third linking pin 19 is equivalent to the point-of-force shaft 43a. It can be understood that the above relationship holds.

On the other hand, a link configuration of the insertion portion 2 for linking the treatment unit 3 and the operating unit 4 exists therebetween.

That is to say, supporting members of the surgical instrument 1 (the first supporting portion 8 and second supporting portion 31) are linked to the first pivot shaft 9 and second pivot shaft 32, across a distance L2 in the direction of the arrow S in the drawing.

The second driving rod 6 and the third driving rod 7 are disposed between the first turning pin 22 and third turning pin 47, and between the second turning pin 23 and fourth turning pin 48, with respective predetermined distances therebetween (preferably set at the same distance as the distance L2).

Also, the first driving rod 5 is disposed so as to have a distance L1, in the direction of the arrow S in the drawing, between the second linking pin 17 and the fourth linking pin 42.

With the present embodiment, the distance between the shafts and fulcrums (pins) is configured as follows.
L1=L2
A1=A3
A1=A2

With regard to the distance A3 and the distance A4, the preferable configuration is that these are substantially the same distance, i.e.,
A3=A4.

According to the first embodiment described above, a link configuration for opening and closing the treatment unit 3 is formed such that a relation, the same as a case wherein the treatment unit 3 is moved in parallel to the side of the operation portion 4 (parallel mapping relation), is realized, so the opening/closing actions of the treatment unit 3 can be easily performed at all times by performing opening/closing actions of the operating unit 4 in arbitrary attitudes which the treatment unit 3 is capable of assuming.

Next, a second embodiment of the present invention will be described below.

The basic configuration of the present embodiment is generally the same as that of the above-described first embodiment, with only the shape of a part of the members differing somewhat. Accordingly, with the present embodiment, the configurations which are the same as those of the above-described first embodiment will be denoted with the same reference numerals and omitted from the drawings, and only the differing parts will be described below in detail.

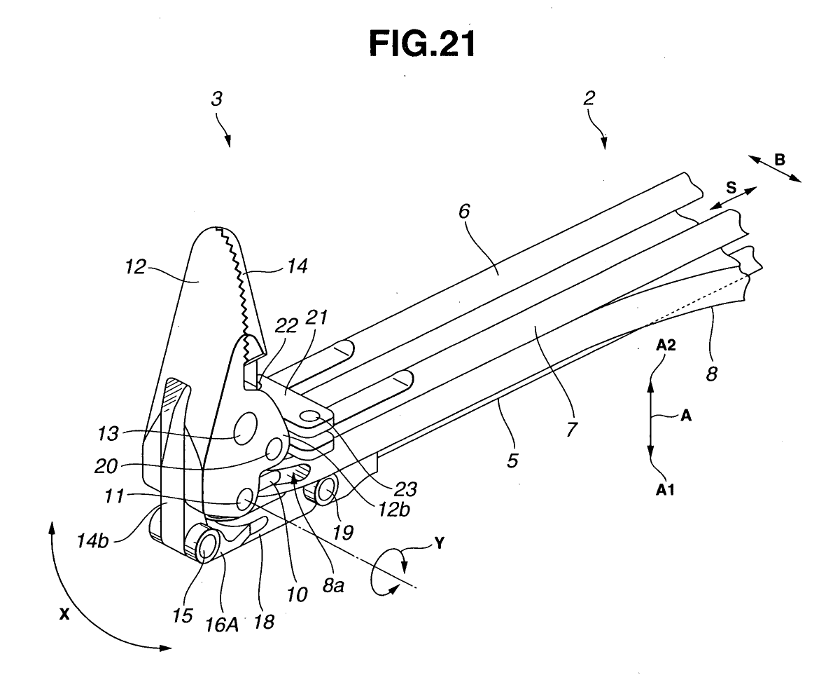

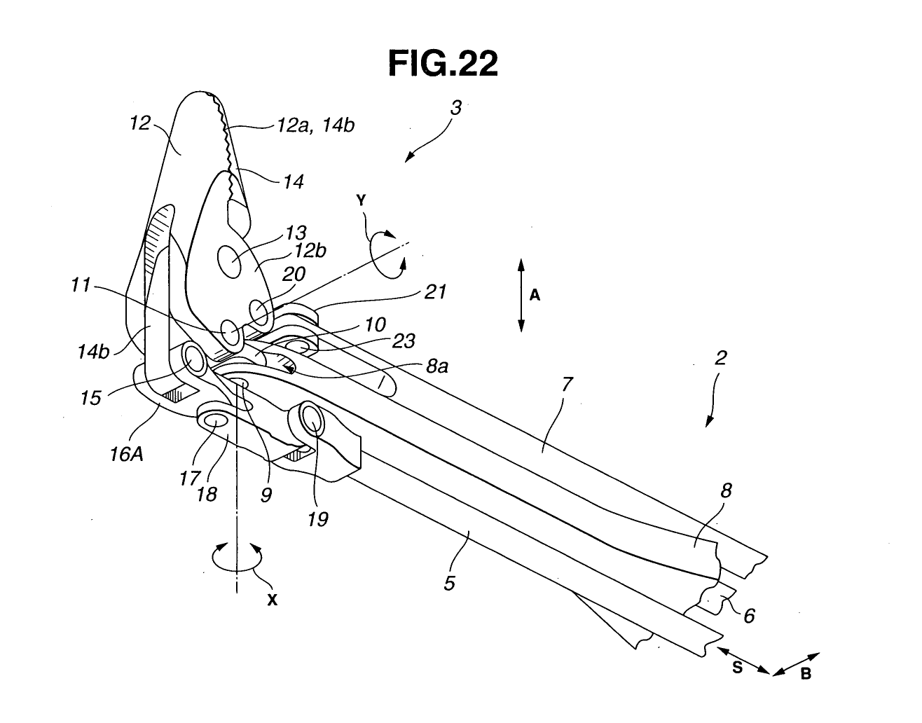

With the surgical instrument (1) according to the present embodiment, the distance in the direction of the arrow S shown in FIG. 21 and FIG. 22 between the first linking pin 15 (see FIG. 21 and FIG. 22) and the second linking pin 17 (see FIG. 22) of the treatment unit 3 (see the distance A1 in FIG. 20) is set so as to be longer than that in the case of the above-described first embodiment. Accordingly, a first linking member 16A of the treatment unit 3 is formed with a shape somewhat different from that of the first linking member 16 of the above-described first embodiment.

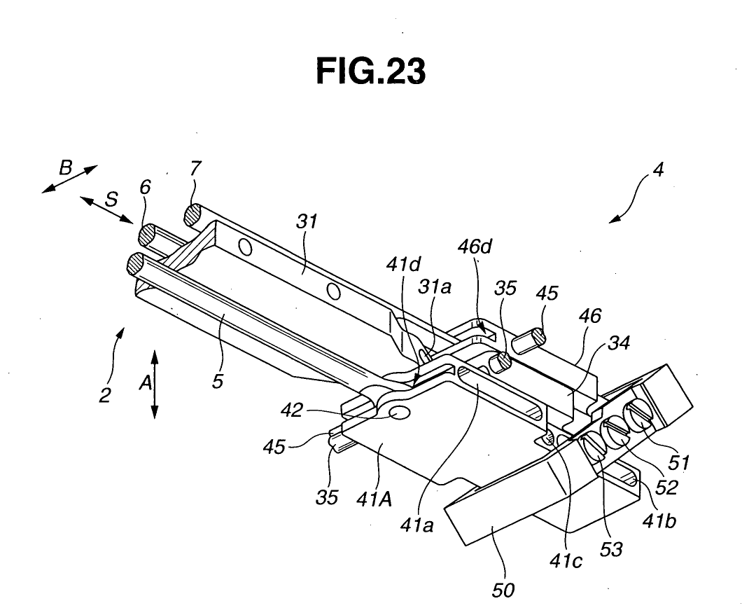

Also, with the operating unit 4 of the surgical instrument (1) according to the present embodiment, the distance between the fourth linking pin 42 (see FIG. 23) and the fifth linking pin 49 (not shown, see FIG. 20), i.e., the distance in the direction of the arrow S in FIG. 23 between the fourth linking pin 42 and the portion at which the first linking pin 15 should be disposed in the event of assuming parallel moving of the treatment unit 3 toward the operating unit 4 (see FIG. 20) (see the distance A2 in FIG. 20) is set so as to be shorter than that in the case of the above-described first embodiment. Accordingly, the shape of the third linking member 41A in the operating unit 4 is formed so as to be somewhat different in comparison with the third linking member 41 of the above-described first embodiment, i.e., such that the position of disposing the fourth linking pin 42 is somewhat different.

The distance in the axial direction S of the insertion portion 2 between the second linking pin 17 of the treatment unit 3 side and the fourth linking pin 42 of the operating unit 4 side (see FIG. 20) is configured so as to have the same distance L1 as with the above-described first embodiment.

More concisely, while the present embodiment is configured such that
L1=L2,
it is configured such that
A1≠A3
A1≠A2.

Also, with regard to the distance A3 and the distance A4, the point that the preferable configuration is that these are substantially the same distance, i.e.,
A3=A4,
is the same as with the above-described first embodiment.

The other configurations are exactly the same as with the above-described first embodiment. Also, the operations of the present embodiment are exactly the same as with the above-described first embodiment.

As described above with the second embodiment, the same advantages as with the above-described first embodiment can be obtained. At the same time, the opening/closing action of the treatment unit 3 and the operating unit 4 can be made smoother in any attitude which the present surgical instrument 1 might assume, accordingly contributing to the operability of the surgical instrument 1 in the use thereof.

Also, employing the link configuration according to the present embodiment allows advantages the same as those of the above-described first embodiment to be obtained even in cases wherein the predetermined members making up the opening/closing link mechanism (opening/closing operating means) of the treatment unit 3, i.e., the parts equivalent to the reference symbol A1 and the reference symbol A3 in FIG. 20, cannot be configured with a relation wherein distance A1 distance A3, due to dimensional restrictions or the like, for example.

Next, a third embodiment of the present invention will be described.

The basic configuration of the present embodiment is generally the same as that of the above-described first embodiment, with only the configuration of a part of the members differing somewhat. Accordingly, with the present embodiment, the configurations which are the same as those of the above-described first embodiment will be denoted with the same reference numerals and omitted from the description and the drawings, and only the differing parts will be described below in detail.

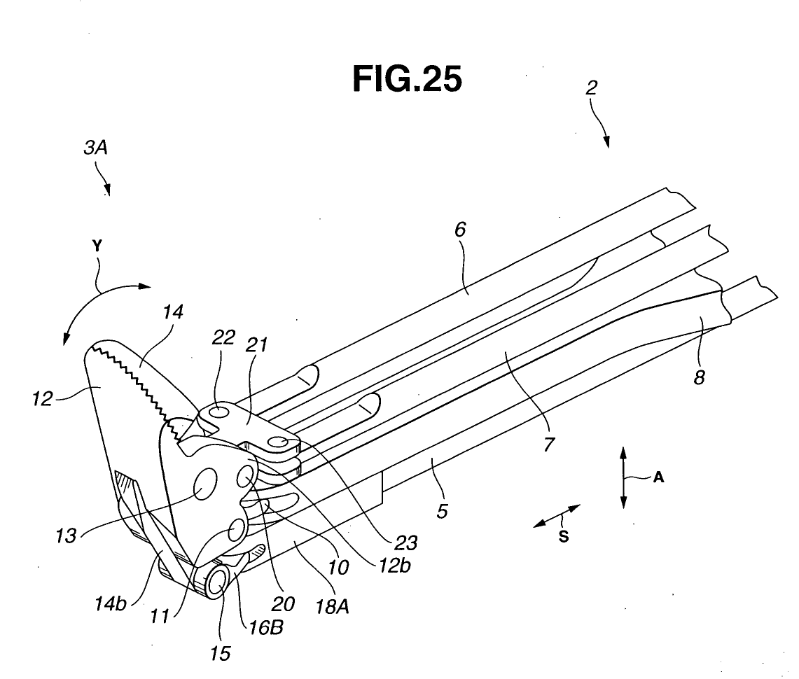

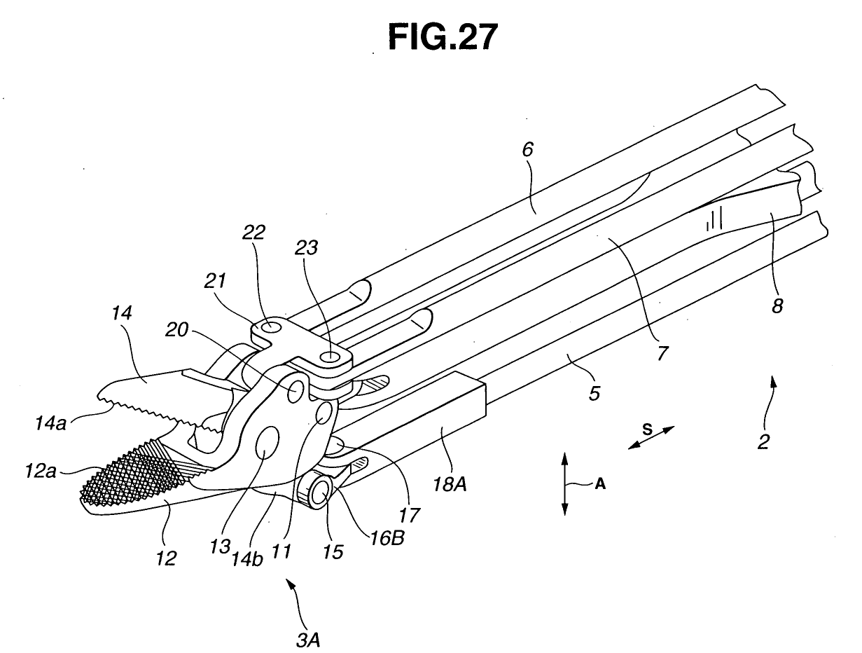

As shown in FIG. 25 through FIG. 27, with the treatment unit 3A of the surgical instrument 1A according to the present embodiment, the configuration is such that the third linking pin 19 (see FIG. 2 through FIG. 4) of the treatment unit 3 of the surgical instrument 1 according to the above-described first embodiment has been eliminated.

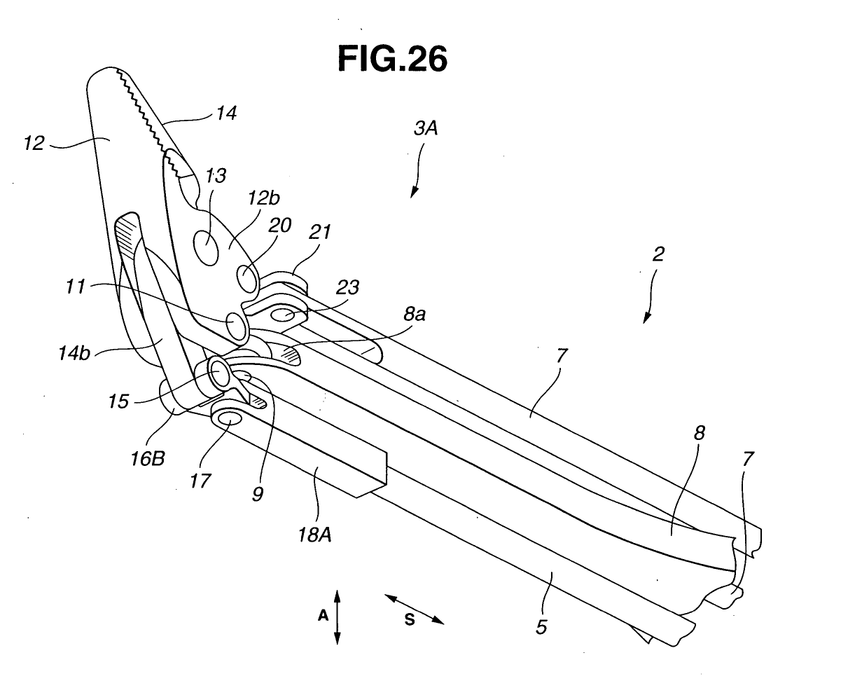

Accordingly, the first linking pin 15 is provided at the tip portion of the arm portion 14b of the base side of the second treatment piece 14, and the one portion of a first linking member 16B is linked by this. Also, the second linking pin 17 (Cannot be shown in FIG. 25. See FIG. 26) is provided to the other end portion of this first linking member 16B, through which one end portion of the second linking member 18A is linked. The other part of this second linking member 18 is directly linked to the first driving rod 5 by predetermined means.

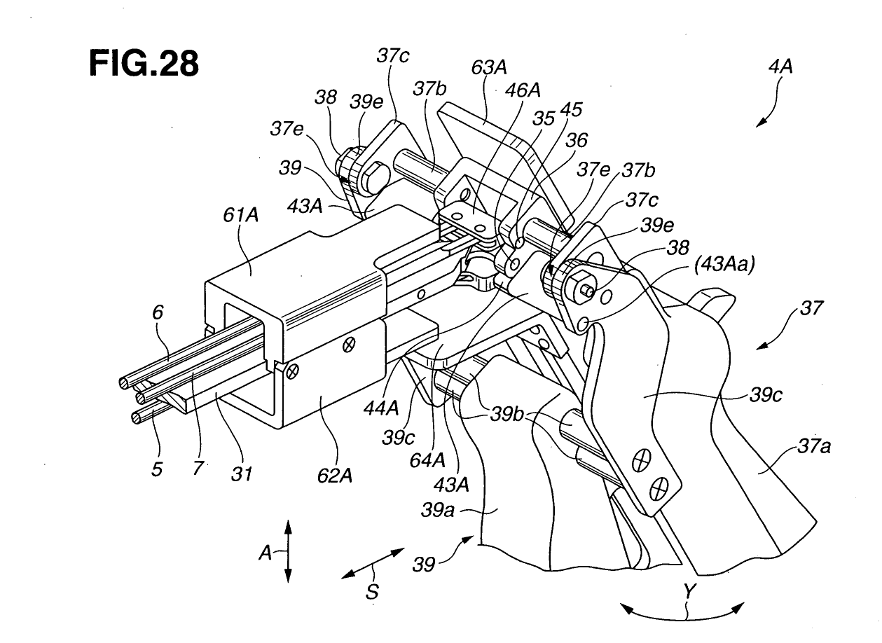

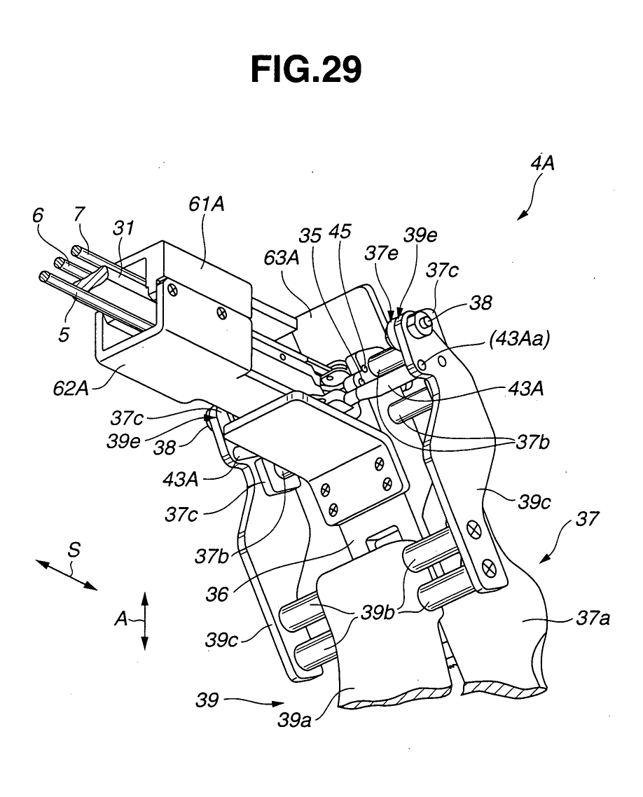

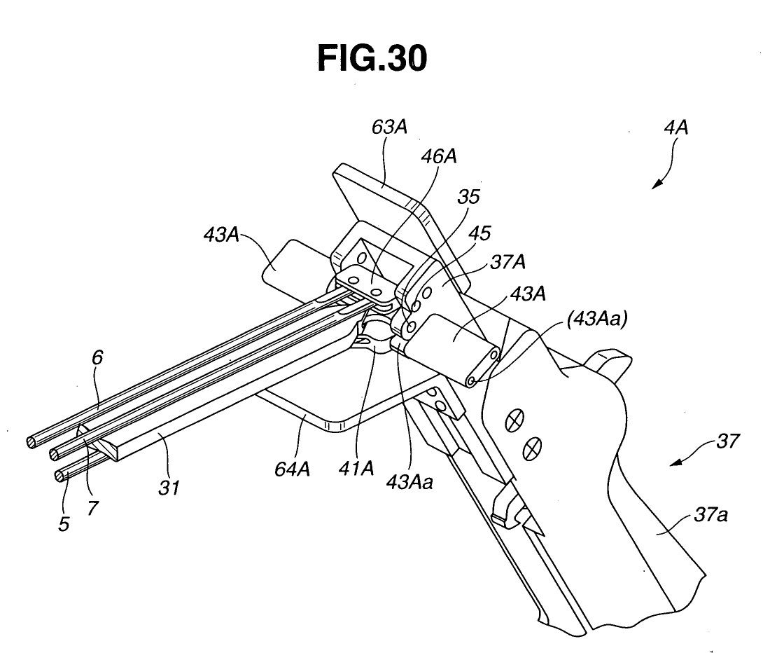

On the other hand, as shown in FIG. 28 through FIG. 32, with the operating unit 4A of the surgical instrument 1A according to the present embodiment, the configuration is such that the fifth linking pin 49 and the fifth linking member 44 in the above-described first embodiment are eliminated. Accordingly, the point-of-force shaft 43a of the fourth linking member 43A is directly linked to the second handle 39A by predetermined means, and the point-of-force shaft 43a fits to the point-of-force shaft receiving hole 41c provided at the side face of the third linking member 41B so as to be capable of turning.

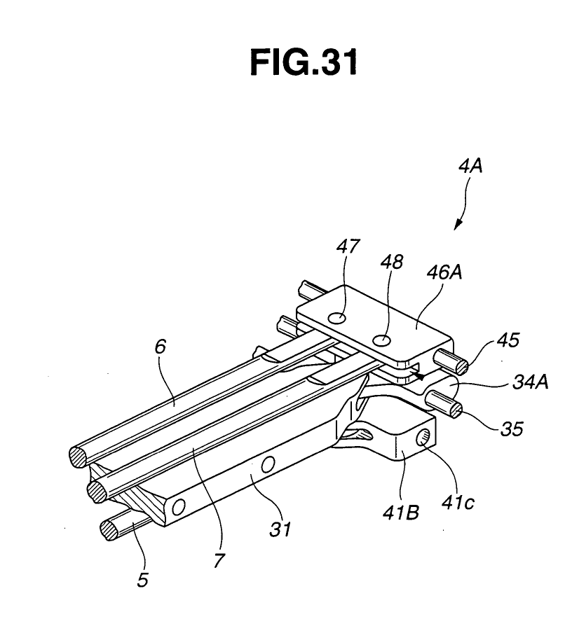

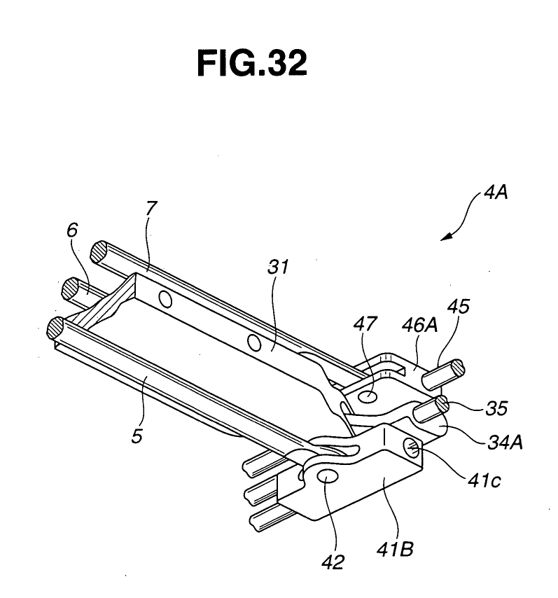

Also, as shown in FIG. 31 and FIG. 32, with the present embodiment, the configuration is such that the cradle 50 and the surrounding components in the above-described first embodiment are eliminated. Accordingly, the third turning plate 34A, the fourth turning plate 46A, and the third linking member 41B, are configured in a form simplified as compared to that of the third turning plate 34, the fourth turning plate 46, and the third linking member 41 according to the above-described first embodiment (see FIG. 8 through FIG. 11, etc.).

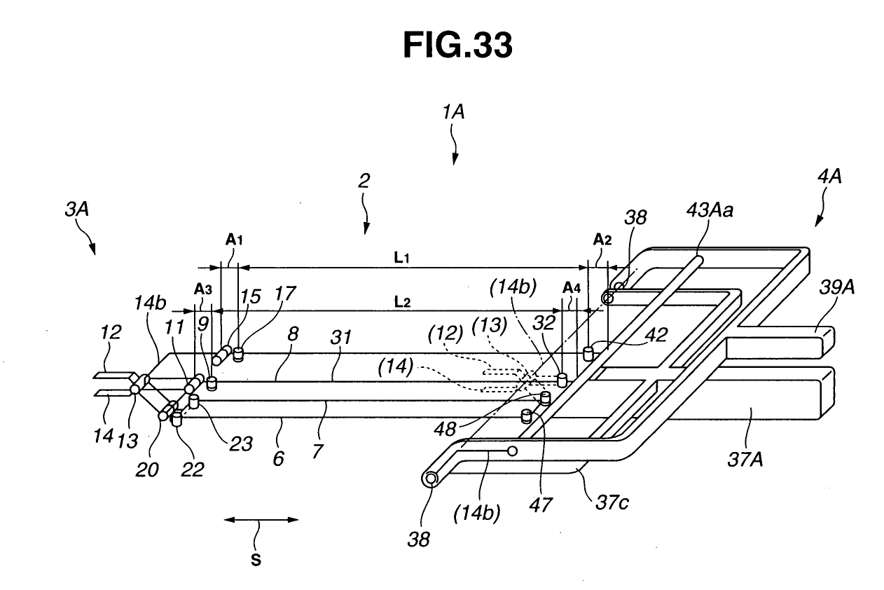

Further, the link configuration of the present embodiment is as shown in FIG. 33. That is, the distance L1 between the second linking pin 17 and the fourth linking pin 42 in the direction of the arrow S shown in the drawing, and the distance L2 between the first pivot shaft 9 and the second pivot shaft 32 in the direction of the arrow S shown in the drawing, is configured such that
L1=L2.

The distance A1 between the first linking pin 15 and the second linking pin 17 in the direction of the arrow S shown in the drawing, and the distance A3 between the first pivot pin 11 and the first pivot shaft 9 in the direction of the arrow S shown in the drawing, is configured such that
A1=A3.

Also, the distance A1 between the first linking pin 15 and the second linking pin 17 in the direction of the arrow S shown in the drawing, and the distance A2 between the fourth linking pin 42 and the point-of-force shaft 43Aa (the portion at which the first linking pin 15 should be disposed in the event of assuming parallel moving of the treatment unit 3A toward the operating unit 4A) in the direction of the arrow S shown in the drawing, is configured such that
A1=A2.

Also, with regard to the distance A3 and the distance A4, the point that the preferable configuration is that these are substantially the same distance, i.e.,
A3=A4,
is the same as with the above-described first embodiment.

The other configurations are exactly the same as with the above-described first embodiment.

The operations of the surgical instrument 1A according to the present embodiment configured in this way will be described below in brief.

With the surgical instrument 1A according to the present embodiment, performing turning operations of the operating unit 4A in the direction of the arrow Y shown in FIG. 28 turns the treatment unit 3A in the direction of the arrow Y shown in FIG. 25 (the vertical direction as to the direction following the axial direction S of the insertion portion 2). FIG. 25 and FIG. 28 illustrate the maximum flexion attitude in this case.

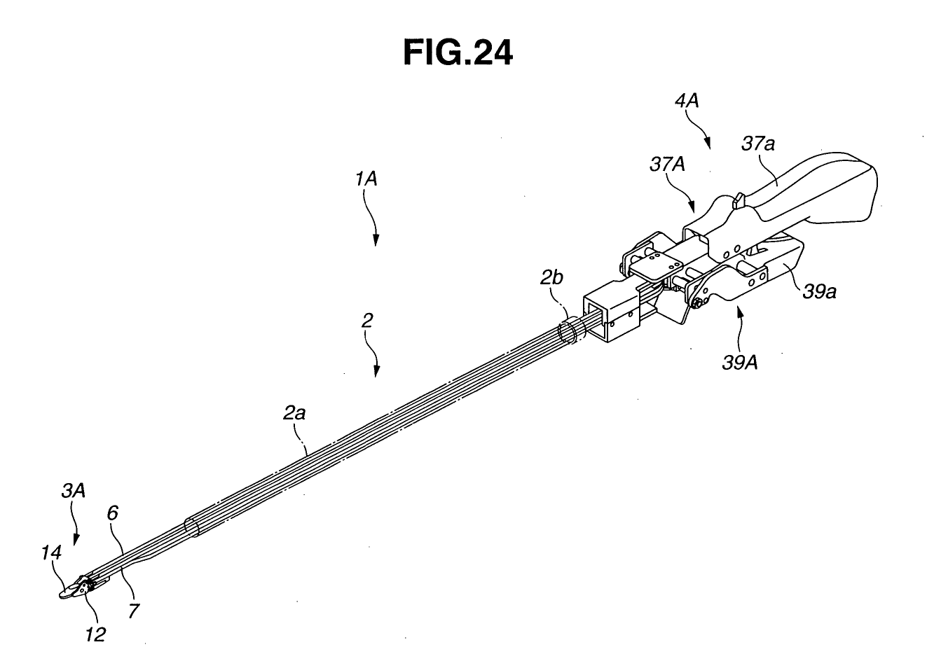

That is, the treatment unit 3A of the surgical instrument 1A according to the present embodiment is capable of turning within a range between a state wherein the treatment unit 3 and operating unit 4 are both horizontal (angle of 0 degrees) as to the insertion portion 2 (see FIG. 24), and a state wherein the treatment unit 3 and operating unit 4 are at an angle of 60 degrees as to the insertion portion 2 (see the maximum flexion attitude shown in FIG. 25 and FIG. 28).

In this way, with the present embodiment, the range of the treatment unit 3A turning in the vertical direction is somewhat narrower with the present embodiment as compared with the above-described first embodiment. This is due to, as described above, eliminating the third linking pin 19 in the above first embodiment and simplifying the configuration. That is to say, because the configuration of the opening/closing mechanism (opening/closing operating means) for opening and closing the treatment unit 3A is situated at a position somewhat moved toward the operating unit 4. However, a sufficient turning range has been secured for using the surgical instrument 1A.

Note that exactly a turning range equal to that of the above-described first embodiment is secured for the turning in the horizontal direction. Also, other operations, such as the opening/closing actions of the treatment unit 3A for example, are exactly the same as with the above-described first embodiment.

According to the third embodiment as described above, advantages similar to those of the above-described first embodiment can be obtained, and further, a generally equal operating range can be secured while simplifying the link configuration for the treatment unit 3A as compared to that of the above-described first embodiment. Thus, this contributes to simplification of the entire device, and also can contribute to reduction in manufacturing costs.

Next, a fourth embodiment of the present invention will be described below.

The basic configuration of the present embodiment is generally the same as that of the above-described third embodiment, with only the shape of a part of the members differing somewhat. Accordingly, with the present embodiment, the configurations which are the same as those of the above-described third embodiment will be denoted with the same reference numerals and omitted from the drawings, and only the differing parts will be described below in detail.

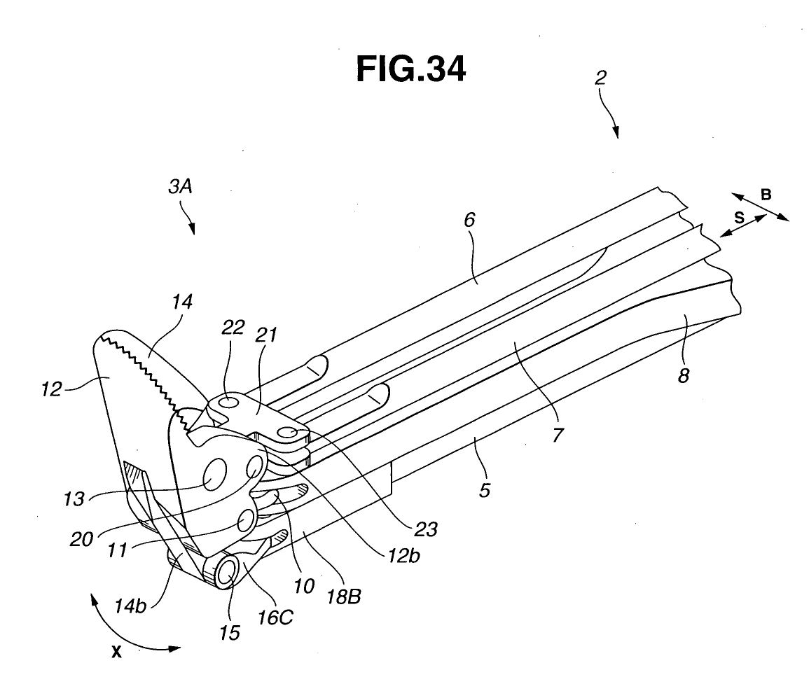

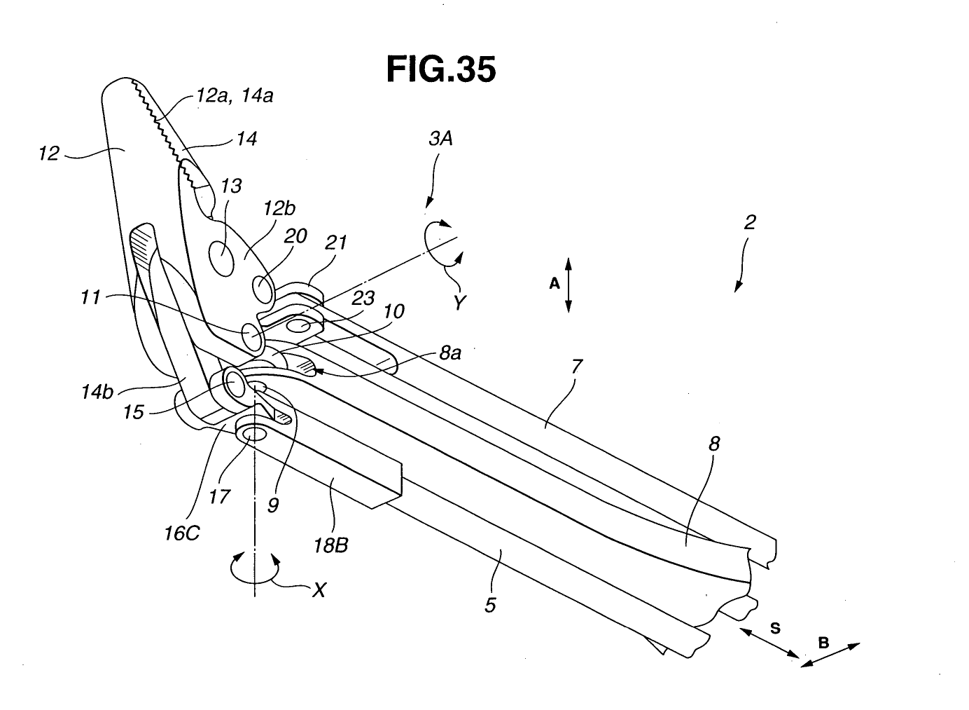

With the surgical instrument (1A) according to the present embodiment, the distance in the direction of the arrow S shown in FIG. 34 and FIG. 35 between the first linking pin 15 (see FIG. 34 and FIG. 35) and the second linking pin 17 (see FIG. 35) of the treatment unit 3A (see the distance A1 in FIG. 33) is set so as to be longer than that in the case of the above-described third embodiment. Accordingly, a first linking member 16C of the treatment unit 3A is formed with a shape somewhat different from that of the first linking member 16B of the above-described third embodiment.

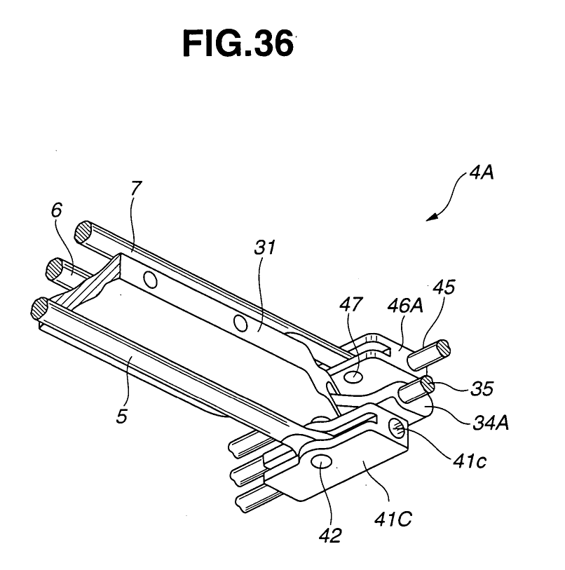

Also, with the operating unit 4A of the surgical instrument (1A) according to the present embodiment, the distance between the fourth linking pin 42 (see FIG. 36) and the portion at which the first linking pin 15 should be disposed in the event of assuming parallel moving of the treatment unit 3A toward the operating unit 4A (see FIG. 33), i.e., the portion equivalent to the point-of-force shaft 43Aa in the direction of the arrow S shown in FIG. 34 and FIG. 35 (see the distance A2 in FIG. 33) is set so as to be shorter than that in the case of the above-described third embodiment. Accordingly, the shape of the third linking member 41B in the operating unit 4B is formed so as to be somewhat different in comparison with the third linking member 41 of the above-described third embodiment, i.e., such that the position of disposing the fourth linking pin 42 is somewhat different.

The distance in the axial direction S of the insertion portion 2 between the second linking pin 17 of the treatment unit 3A side and the fourth linking pin 42 of the operating unit 4A side (see FIG. 33) is configured so as to have the same distance L1 as with the above-described third embodiment.

More concisely, as with the third embodiment, while the present embodiment is configured such that
L1=L2,
it is configured such that
A1≠A3
A1≠A2.

Also, with regard to the distance A3 and the distance A4, the point that the preferable configuration is that these are substantially the same distance, i.e.,
A3=A4,
is the same as with the above-described third embodiment. The other configurations are exactly the same as with the above-described third embodiment.

Further, the operations of the surgical instrument (1A) according to the present embodiment are exactly the same as with the above-described third embodiment.

As described above, the fourth embodiment can contribute to reduction of manufacturing costs by realizing simplification of the device as with the above-described third embodiment.

Also, employing the link configuration according to the present embodiment allows advantages the same as those of the above-described third embodiment to be obtained even in cases wherein the predetermined members making up the opening/closing link mechanism (opening/closing operating means) of the treatment unit 3A, i.e., the parts equivalent to the reference symbol A1 and the reference symbol A3 in FIG. 33, cannot be configured with a relation wherein distance A1=distance A3, due to dimensional restrictions or the like, for example.

Next, a fifth embodiment of the present invention will be described.

The basic configuration of the present embodiment is generally the same as that of the above-described first and second embodiments, with only the form of the link configuration being different.

Accordingly, with the present embodiment, detailed description and illustration in the drawings of the configuration of the device itself will be omitted, and only the differing parts will be described below in detail. Note that the same components as those of the above-described first and second embodiment will be denoted with the same reference numerals in disclosing the components and so forth.

With the present embodiment, the opening/closing link mechanism (opening/closing operating means) for opening and closing the treatment unit 3B is as follows. That is to say, the second linking pin 17 is provided at the base side of the first linking pin 15 provided at the base portion of the treatment unit 3B, and the third linking pin 19 is provided at the base side of this second linking pin 17.

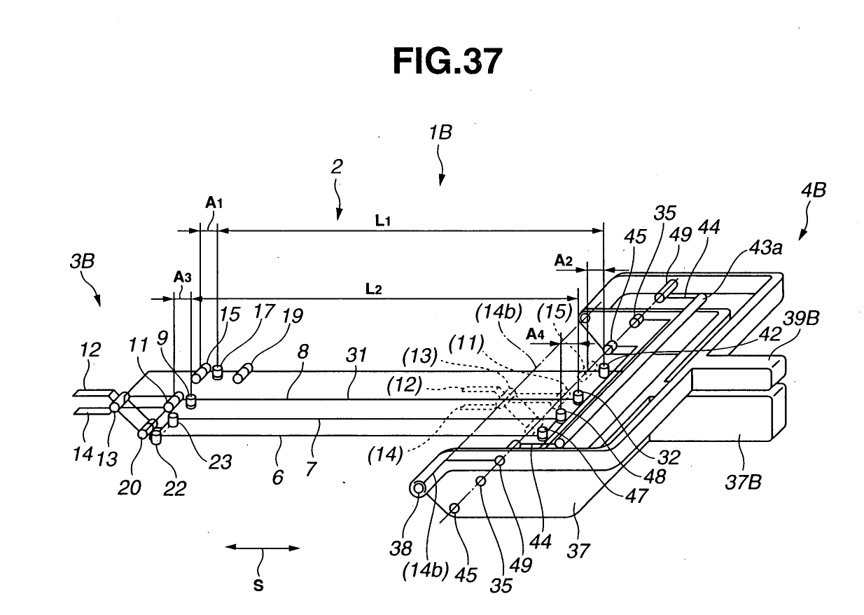

On the other hand, at the tip side of the operating unit 4B, the fifth linking pin 49 is provided at the portion at which the first linking pin 15 should be disposed in the event of assuming parallel moving of the treatment unit 3B toward the operating unit 4B (see the dotted line in FIG. 37), and the fourth linking pin 42 is provided at the base side of the fifth linking pin 49. At the base side of this fourth linking pin 42, the point-of-force shaft 43a is positioned at the portion at which the third linking pin 19 should be disposed in the event of assuming parallel moving of the treatment unit 3B toward the operating unit 4B.

Also, the turning link mechanism for turning the treatment unit 3B is as follows. That is, the first pivot shaft 9 is provided at the base side of the first pivot pin 11 provided at the base side of the treatment unit 3B, and the first turning pin 22 and second turning pin 23 are provided at the base side of the second pivot pin 20.

On the other hand, at the tip side of the operating unit 4B, the third pivot pin 35 is provided at the portion at which the first pivot pin 11 should be disposed in the event of assuming parallel moving of the treatment unit 3B toward the operating unit 4B, and the second pivot shaft 32 is provided to the base side of the third pivot pin 35. Also, in the same way, the fourth pivot pin 45 is provided at the portion at which the second pivot pin 20 should be disposed in the event of assuming parallel moving of the treatment unit 3B toward the operating unit 4B, and the third turning pin 47 and fourth turning pin 48 are provided to the base side of the fourth pivot pin 45.

With the present embodiment, the distance L1 between the second linking pin 17 and the fourth linking pin 42 in the direction of the arrow S shown in the drawing, and the distance L2 between the first pivot shaft 9 and the second pivot shaft 32 in the direction of the arrow S shown in the drawing, is configured such that
L1=L2.

Also, the distance A1 between the first linking pin 15 and the second linking pin 17 in the direction of the arrow S shown in the drawing, and the distance A3 between the first pivot pin 11 and the first pivot shaft 9 in the direction of the arrow S shown in the drawing, is configured such that, as with the first embodiment,
A1=A3.

Further, the distance A1 between the first linking pin 15 and the second linking pin 17 in the direction of the arrow S shown in the drawing, and the distance A2 between the fourth linking pin 42 and the fifth linking pin 49 (the portion at which the first linking pin 15 should be disposed in the event of assuming parallel moving of the treatment unit 3B toward the operating unit 4B) in the direction of the arrow S shown in the drawing, is configured such that
A1=A2.

Note that with regard to the distance A3 and the distance A4, the point that the preferable configuration is that these are substantially the same distance, i.e.,
A3=A4,
is the same as with the above-described first embodiment.

The other configurations are exactly the same as with the above-described first embodiment. Also, the operations of the present invention are also exactly the same as with the above-described first embodiment.

With the fifth embodiment as described above, advantages the same as with the above-described first embodiment can be obtained. Further, a link configuration for opening and closing the treatment unit 3 and a link configuration for turning the treatment unit 3 have been formed to realize the same relation as with the case of assuming parallel moving of the treatment unit 3 toward the operating unit 4, so opening/closing operations and turning operations of the treatment unit 3 can be easily performed at all times by performing opening/closing operations and turning operations of the operating unit 4, in any arbitrary attitude which the treatment unit 3 is capable of assuming.

Note that with the above-described fifth embodiment, the configuration may be made in the same way as with the above-described second and fourth embodiments such that
L1=L2
A1≠A3
A1≠A2.

In this case as well, with regard to the distance A3 and the distance A4, the point that the preferable configuration is that these are substantially the same distance, i.e.,
Distance A3=Distance A4,
is the same.

The operations of this configuration are also the same as with the above-described second and fourth embodiments.

It is clear that a wide range of embodiments can be made based on this invention, without departing from the spirit and scope of the invention. This invention is not restricted by any particular embodiment, and is only restricted by the appended claims.

1. A surgical instrument comprising:
an insertion portion; a treatment unit which is provided on the tip portion of the insertion portion and which is capable of turning as to the shaft of the insertion portion and capable of opening and closing; an operating unit provided at the base portion of the insertion portion and which is capable of turning as to the shaft of the insertion portion and capable of opening and closing; a supporting member linking the treatment unit and the operating unit; and a plurality of driving rods arrayed in parallel or generally in parallel with the supporting member, so as to be capable of advancing and retreating;
wherein the opening/closing operating means of the treatment unit and the opening/closing operating means of the operating unit are in a parallel mapping relation following the insertion portion.

2. A surgical instrument according to claim 1, wherein the distance between two joints, provided at the tip side and base side of at least one driving rod of the driving rods, and having axes in a direction perpendicular to opening/closing shafts of the treatment unit, and the distance between two turning shafts, provided at the tip side and base side of the supporting member, and having axes in a direction perpendicular to opening/closing shafts of the treatment unit, are configured so as to be the same length.

3. A surgical instrument according to claim 1, wherein the treatment unit comprises a turning treatment piece capable of turning as to the supporting member, and an opening/closing treatment piece linked to the turning treatment piece through a first opening/closing shaft so as to be capable of opening and closing;
and wherein the distance between a first joint provided to the base portion of the opening/closing treatment piece and a second joint adjacent to the base side of the first joint, and the distance between a first turning shaft provided to the base portion of the turning treatment piece and a second turning shaft adjacent to the base side of the first turning shaft, are configured so as to be the same length.

4. A surgical instrument according to claim 2, wherein the treatment unit comprises a turning treatment piece capable of turning as to the supporting member, and an opening/closing treatment piece linked to the turning treatment piece through a first opening/closing shaft so as to be capable of opening and closing;
and wherein the distance between a first joint provided to the base portion of the opening/closing treatment piece and a second joint adjacent to the base side of the first joint, and the distance between a first turning shaft provided to the base portion of the turning treatment piece and a second turning shaft adjacent to the base side of the first turning shaft, are configured so as to be the same length.

5. A surgical instrument according to claim 1, wherein the operating unit comprises a turning operating piece capable of turning as to the supporting member, and an opening/closing operating piece linked to the turning operating piece through a second opening/closing shaft so as to be capable of opening and closing;
and wherein the longitudinal distance between a third joint provided to the opening/closing operating piece and a fourth joint provided on the driving rod essentially adjacent to the third joint, and the distance between the first joint provided to the base portion of the opening/closing treatment piece and a second joint adjacent to the base side of the first joint, are configured so as to be the same length.

6. A surgical instrument according to claim 2, wherein the operating unit comprises a turning operating piece capable of turning as to the supporting member, and an opening/closing operating piece linked to the turning operating piece through a second opening/closing shaft so as to be capable of opening and closing;
and wherein the longitudinal distance between a third joint provided to the opening/closing operating piece and a fourth joint provided on the driving rod essentially adjacent to the third joint, and the distance between the first joint provided to the base portion of the opening/closing treatment piece and a second joint adjacent to the base side of the first joint, are configured so as to be the same length.

7. A surgical instrument according to claim 3, wherein the operating unit comprises a turning operating piece capable of turning as to the supporting member, and an opening/closing operating piece linked to the turning operating piece through a second opening/closing shaft so as to be capable of opening and closing;
and wherein the longitudinal distance between a third joint provided to the opening/closing operating piece and a fourth joint provided on the driving rod essentially adjacent to the third joint, and the distance between the first joint provided to the base portion of the opening/closing treatment piece and a second joint adjacent to the base side of the first joint, are configured so as to be the same length.
